(19) United States
(12) Patent Application Publication
(10) Pub. No.: US 2012/0196274 A1
(43) Pub. Date: Aug. 2, 2012

(54) MATERIALS AND METHODS FOR GENOTYPING AND QUANTIFYING A HIGH-RISK HUMAN PAPILLOMAVIRUS

(75) Inventors: Sameera RANGWALA, Gaithersburg, MD (US); Lori KOBAYASHI, Boonsboro, MD (US); Tanya GAY, Gaithersburg, MD (US)

(73) Assignee: QIAGEN GAITHERSBURG, INC., Gaithersburg, MD (US)

(21) Appl. No.: 13/346,550

(22) Filed: Jan. 9, 2012

Related U.S. Application Data
(60) Provisional application No. 61/430,797, filed on Jan. 7, 2011.

(51) Int. Cl. C12Q 1/70; C07H 21/00

(52) U.S. Cl. 435/5; 536/24.32; 536/24.33

(57) ABSTRACT

Nucleic acids, assays, and methods for the detection and quantification of high risk HPV types are disclosed.

### CROSS REFERENCE TO RELATED APPLICATIONS

This application claims priority to U.S. Provisional Application No. 61/430,797, filed on Jan. 7, 2011, which is hereby incorporated by reference in its entirety.

### FIELD OF INVENTION

The present invention relates to assays, methods, reagents, systems, and kits for determining the presence of a nucleic acid in a sample. More specifically, the invention relates to the detection, genotyping, and quantification of a high-risk human papillomavirus in a sample.

### BACKGROUND

Persistent infection with oncogenic high-risk (HR) human papillomavirus (HPV) genotypes is associated with increased risk for the presence of developing high-grade dysplasia or cervical carcinoma, vulvar or vaginal carcinoma. There are approximately 18 HR-HPV types that infect the genital mucosa and are defined as carcinogenic. Of these HPV types 16, 18, and 45 are associated with the highest rate of cervical adenocarcinomas. HPV types 16 and 18 account for 70% of all cervical cancers, are the most prevalent carcinogenic HPV types, and are targeted by the new HPV vaccines.

It is well known that screening programs that utilize molecular screening for the presence of HR-HPV, either independently or as an adjunct to cytological testing, improve the efficacy for standard of care processes. Moreover, tests that quantify viral load may also be important, as increased HPV load has been shown to correlate with disease progression in cervical cancer. However, the predictive value of calculating HPV DNA load may vary depending upon the type of HPV infection present, since the biological behavior of the various HR-HPV types differ. Therefore, HR-HPV load can be a type-dependent risk marker for invasive carcinoma. As such, the ability to accurately genotype an HPV infection and quantify the viral load would be a valuable tool.

Unfortunately, the majority of HR-HPV screening methods do not genotype for individual HPV types, but rather detect an entire group of HR-HPV. Some PCR-based assays do exist for genotyping high risk HPV types, based on amplifying a portion of the L1 region various HR-HPV genomes. However, tests targeting the L1 region are prone to false negative results because part of the L1 open reading frame is lost in a low percentage of integration events into human genome. As such, use of an L1-based HR-HPV screening program may result in the delayed monitoring and treatment of potential high risk HPV infections. Accordingly, a need exists for materials and methods for accurately genotype and/or quantifying a HR-HPV infection based on a region of the HPV genome other than L1 would be a valuable tool.

### SUMMARY

Compositions, nucleic acids, assays, kits, and methods described herein address the shortcomings described above by providing nucleic acids adapted to specifically detect the E6/E7 region of high risk HPV types.

In an aspect, a nucleic acid primer or probe specific for a target sequence in the E6/E7 region of a high-risk HPV genome, wherein: a. said nucleic acid primer or probe has 80% identity or greater across its entire length to both the target sequence and at least one nucleic acid sequence in E6/E7 region of a genome of a subtype thereof; and b. said nucleic acid primer or probe does not hybridize to a nucleic acid derived from a different HPV type.

In one embodiment, the high risk HPV genome is selected from the group consisting of HPV 16, HPV 18, and HPV 45

In one embodiment, the nucleic acid primer comprises, consists essentially of, or consists of a sequence having about 70% to 100% identity with a sequence selected from the group consisting of SEQ ID NO:1 to SEQ ID NO:12 and SEQ ID NO:16 to SEQ ID NO:21, and/or a complement thereof.

In one embodiment, the nucleic acid probe comprises, consists essentially of, or consists of a sequence having about 70% to 100% identity with a sequence selected from the group consisting of SEQ ID NO:3, SEQ ID NO:6, SEQ ID NO:9, SEQ ID NO:12, SEQ ID NO:18, and SEQ ID NO:21, and/or a complement thereof.

In one embodiment, the primer is capable of amplifying a portion of the E6/E7 region of the HPV 16 genome, but not an E6/E7 region of HPV 18 or HPV 45 genome.

In one embodiment, the nucleic acid primer comprises, consists essentially of, or consists of SEQ ID NO:1 or SEQ ID NO:2 or a complement thereof.

In one embodiment, the primer is capable of amplifying a portion of the E6/E7 region of the HPV 18 genome, but not an E6/E7 region of HPV 16 or HPV 45 genome.

In one embodiment, the nucleic acid primer comprises, consists essentially of, or consists of SEQ ID NO:4 or SEQ ID NO:5.

In one embodiment, the primer is capable of amplifying a portion of the E6/E7 region of the HPV 45 genome, but not an E6/E7 region of HPV 16 or HPV 18 genome

In one embodiment, the nucleic acid primer of comprises, consists essentially of, or consists of SEQ ID NO:7 or SEQ ID NO:8.

In one embodiment, a primer set is provided comprising at least one nucleic acid primer comprising, consisting essentially of, or consisting of a sequence having about 70% to 100% identity with a sequence selected from the group consisting of SEQ ID NO:1 to SEQ ID NO:12 and SEQ ID NO:16 to SEQ ID NO:21, and/or a complement thereof.

In one embodiment, the primer set comprises, consists essentially of, or consists of at least one primer pair selected from the group consisting of: (a) a primer pair capable of amplifying a portion of the E6/E7 region of the HPV 16 genome, but not an E6/E7 region of HPV 18 or HPV 45 genome; (b) a primer pair capable of amplifying a portion of the E6/E7 region of the HPV 18 genome, but not an E6/E7 region of HPV 16 or HPV 45 genome; and (c) a primer pair capable of amplifying a portion of the E6/E7 region of the HPV 45 genome, but not an E6/E7 region of HPV 16 or HPV 18 genome.

In one embodiment, the primer set comprises, consists essentially of, or consists of a primer pair selected from the group consisting of: (a) a first primer having about 70% to 100% identity with SEQ ID NO: 1 and a second primer having about 70% to 100% identity SEQ ID NO: 2, (b) a first primer having about 70% to 100% identity with SEQ ID NO: 4 and a second primer having about 70% to 100% identity SEQ ID NO: 5, (c) a first primer having about 70% to 100% identity with SEQ ID NO: 7 and a second primer having about 70% to 100% identity SEQ ID NO: 8, (d) a first primer having about 70% to 100% identity with SEQ ID NO: 10 and a second primer having about 70% to 100% identity SEQ ID NO: 11, (e) a first primer having about 70% to 100% identity with SEQ ID NO: 16 and a second primer having about 70% to 100% identity SEQ ID NO: 17, (f) a first primer having about 70% to 100% identity with SEQ ID NO: 19 and a second primer having about 70% to 100% identity SEQ ID NO: 20. The primer set according to claim 13 comprising a primer pair selected from the group consisting of: (a) a first primer consisting of SEQ ID NO: 1 and a second primer having about 70% to 100% identity SEQ ID NO: 2, (b) a first primer consisting of SEQ ID NO: 4 and a second primer having about 70% to 100% identity SEQ ID NO: 5, (c) a first primer consisting of SEQ ID NO: 7 and a second primer having about 70% to 100% identity SEQ ID NO: 8, (d) a first primer consisting of SEQ ID NO: 10 and a second primer having about 70% to 100% identity SEQ ID NO: 11, (e) a first primer consisting of SEQ ID NO: 16 and a second primer having about 70% to 100% identity SEQ ID NO: 17, (f) a first primer consisting of SEQ ID NO: 19 and a second primer having about 70% to 100% identity SEQ ID NO: 20.

In another aspect, a kit for the detection of HPV 16, 18, and/or 45 is provided, said kit comprising a nucleic acid primer or probe specific for a target sequence in the E6/E7 region of a high-risk HPV genome, wherein: a. said nucleic acid primer or probe has 80% identity or greater across its entire length to both the target sequence and at least one nucleic acid sequence in E6/E7 region of a genome of a subtype thereof; and b. said nucleic acid primer or probe does not hybridize to a nucleic acid derived from a different HPV type.

In one embodiment, the kit comprises, consists essentially of, or consists of at least one nucleic acid primer and at least one nucleic acid probe, wherein: (a) said nucleic acid primer comprises, consists essentially of, or consists of a sequence having about 70% to 100% identity with a sequence selected from the group consisting of SEQ ID NO:1, SEQ ID NO:2, SEQ ID NO:4, SEQ ID NO:5, SEQ ID NO:7, SEQ ID NO:8, SEQ ID NO:10, SEQ ID NO:11, SEQ ID NO:16, SEQ ID NO:17, SEQ ID NO:19, and SEQ ID NO:20, or a complement thereof; and (b) said nucleic acid probe comprises, consists essentially of, or consists of a sequence having about 70% to 100% identity with a sequence selected from the group consisting of SEQ ID NO:3, SEQ ID NO:6, SEQ ID NO:9, SEQ ID NO:12, SEQ ID NO:18, and SEQ ID NO:21, or a complement thereof.

In one embodiment, the kit comprises, consists essentially of, or consists of an HPV 16 primer and probe set, an HPV 18 primer and probe set, and/or an HPV 45 primer and probe set.

In one embodiment, (a) the HPV 16 primer and probe set comprises: (i) a primer having 70% to 100% identity with a sequence of SEQ ID NO:1, (ii) a primer having 70% to 100% identity with a sequence of SEQ ID NO:2, and, (iii) a probe having 70% to 100% identity with a sequence of SEQ ID NO:3; (b) the HPV 18 primer and probe set comprises: (i) a primer having 70% to 100% identity with a sequence of SEQ ID NO:4, (ii) a primer having 70% to 100% identity with a sequence of SEQ ID NO:5, and, (iii) a probe having 70% to 100% identity with a sequence of SEQ ID NO:6; and (c) the HPV 45 primer and probe set comprises (i) a primer having 70% to 100% identity with a sequence of SEQ ID NO:7, (ii) a primer having 70% to 100% identity with a sequence of SEQ ID NO:8 and, (iii) a probe having 70% to 100% identity with a sequence of SEQ ID NO:9.

In one embodiment, the kit further comprises a control primer and probe set. In one embodiment, the control primer and probe set comprises (a) a primer having 70% to 100% identity with a sequence of SEQ ID NO:16, (b) a primer having 70% to 100% identity with a sequence of SEQ ID NO:17, and, (c) a probe having 70% to 100% identity with a sequence of SEQ ID NO:18.

In another aspect, a method of genotyping a high-risk HPV is provided, the method comprising: (a) hybridizing at least one nucleic acid primer or probe according to claim 1 to at least a portion of a target sequence in an E6/E7 region of a first high-risk HPV genome; and (b) detecting hybridization of the nucleic acid to the E6/E7 region of the first high-risk HPV genome.

In an embodiment, the nucleic acid primer or probe comprises, consists essentially of, or consists of a sequence having about 70% to 100% identity to a sequence selected from the group consisting of SEQ ID NO:1 to SEQ ID NO:12 and SEQ ID NO:16 to SEQ ID NO:21, and/or a complement thereof.

In an embodiment: (a) a nucleic acid primer is hybridized to the target sequence; and (b) hybridization is detected by a method comprising performing an amplification reaction.

In an embodiment: (a) the high-risk HPV is selected from the group consisting of HPV 16, HPV 18, and HPV 45; and (b) the amplification reaction generates an amplicon corresponding to a portion of the E6/E7 region of the HPV genome.

The method of claim 25 wherein the nucleic acid primer has about 70% to 100% identity across its entire length to a nucleotide sequence selected from the group consisting of: SEQ ID NO: 1; SEQ ID NO: 2; SEQ ID NO: 4; SEQ ID NO: 5; SEQ ID NO: 7; and SEQ ID NO: 8 or a complement thereof.

The method of claim 22, wherein a nucleic acid probe is hybridized to the target sequence, which said nucleic acid probe is optionally detectably labeled.

The method of claim 22, wherein the said method is performed in a real time multiplex PCR format.

The method of claim 28, wherein said method is capable of distinguishing between HPV 16, HPV 18, and HPV 45 infections, said method comprising: (a) providing a sample suspected of comprising a high-risk HPV; (b) contacting the sample with: (i) a primer set capable of amplifying a portion of a target sequence of an E6/E7 region of the HPV 16 genome, but not an E6/E7 region of an HPV 18 or HPV 45 genome; (ii) a primer set capable of amplifying a portion of a target sequence of an E6/E7 region of the HPV 18 genome, but not an E6/E7 region of an HPV 16 or HPV 45 genome; (iii) a primer set capable of amplifying a portion of a target sequence of an E6/E7 region of the HPV 45 genome, but not an E6/E7 region of an HPV 16 or HPV 18 genome; (iv) a detectably labeled nucleic acid probe capable of hybridizing to a portion of the target sequence of the E6/E7 region of the HPV 16 genome, but not an E6/E7 region of an HPV 18 or HPV 45 genome; (v) a detectably labeled nucleic acid probe capable of hybridizing to a portion of the target sequence of the E6/E7 region of the HPV 18 genome, but not an E6/E7 region of an HPV 16 or HPV 45 genome; and (vi) a detectably labeled nucleic acid probe capable of hybridizing to a portion the target sequence of the E6/E7 region of the HPV 45 genome, but not an E6/E7 region of an HPV 16 or HPV 18 genome, wherein each nucleic acid probe has a different detectable label; (c) performing a nucleic acid amplification; and (d) detecting the presence or absence of each detectable label.

In another embodiment, the method is performed on a platform that is capable of replicating a method performed by QIAGEN's Rotor-Gene PCR instrument.

In another embodiment, the method is unaffected by the presence or absence of PreservCyt® (Hologic, Bedford, Mass.) and SurePath™ (Becton Dickinson, Sparks Md.).

In another aspect, a composition is provided comprising a nucleic acid primer or probe as described herein hybridized to a target sequence in the E6/E7 region of a high-risk HPV genome.

In one embodiment, the composition comprises a high-risk HPV genome selected from the group consisting of HPV 16, HPV 18, and HPV 45.

In another embodiment, the composition comprises a primer or probe comprising, consisting essentially of, or consisting of a sequence having 70% to 100% identity with a sequence selected from the group consisting of SEQ ID NO:1 to SEQ ID NO:12 and SEQ ID NO:16 to SEQ ID NO:21, and/or a complement thereof.

### BRIEF DESCRIPTION OF THE DRAWINGS

FIG. 1 illustrates assay sensitivity of an exemplary HPV primer/probe set.

FIG. 2 illustrates the results of a multiplex suppression study for exemplary primer/probe sets.

FIG. 3 illustrates exemplary HPV 16 detections in both singleplex and multiplex detection assays.

FIG. 4 illustrates exemplary HPV 16 detections in both singleplex and multiplex detection assays.

FIG. 5 illustrates exemplary HPV 16 detections in both singleplex and multiplex detection assays.

FIG. 6 illustrates the results of cycling orange (ROX) in an exemplary multiplex assay and the specific detection of HPV 16 at 10³ copies/reaction and 10⁵ copies/reaction.

FIG. 7 illustrates the results (CT values) of cycling orange (ROX) in an exemplary multiplex assay and the detection of HPV 16.

FIG. 8 illustrates the results of cycling yellow (HEX) in an exemplary multiplex assay and the specific detection of HPV 18 at 10³ copies/reaction and 10⁵ copies/reaction.

FIG. 9 illustrates the results (CT values) of cycling orange (HEX) in an exemplary multiplex assay and the detection of HPV 18.

FIG. 10 illustrates the results of cycling green (FAM) in an exemplary multiplex assay and the specific detection of HPV 45 at 10³ copies/reaction and 10⁵ copies/reaction.

FIG. 11 illustrates the results (CT values) of cycling orange (FAM) in an exemplary multiplex assay and the detection of HPV 45.

FIG. 12 illustrates the results of cycling red (Cy5) in an exemplary multiplex assay and the specific detection of the beta-globin as a control.

FIG. 13 illustrates the results (CT values) of cycling orange (Cy5) in an exemplary multiplex assay and the detection of the control, beta-globin.

FIG. 14 illustrates the results of an exemplary multiplex assay and the detection of HPV 16 at 5, 10, 100, and 10³ copies/reaction.

FIG. 15 illustrates the results (CT values) of cycling orange (ROX) in an exemplary multiplex assay and the detection of HPV 16 at varying copies/reaction.

FIG. 16 illustrates the results of an exemplary multiplex assay and the detection of HPV 18 at 5, 10, 100, and 10³ copies/reaction.

FIG. 17 illustrates the results (CT values) of cycling yellow (HEX) in an exemplary multiplex assay and the detection of HPV 18 at varying copies/reaction.

FIG. 18 illustrates the results of an exemplary multiplex assay and the detection of HPV 45 at 5, 10, 100, and 10³ copies/reaction.

FIG. 19 illustrates the results (CT values) of cycling green (FAM) in an exemplary multiplex assay and the detection of HPV 45 at varying copies/reaction.

### DETAILED DESCRIPTION

The present disclosure includes methods, compositions, reagents, systems, and kits for rapidly determining the presence of a nucleic acid molecule in a sample and for quantifying the target nucleic acid molecule. The methods, compositions, reagents, systems, and kits may be used for clinical diagnostic purposes, and/or for characterizing HPV viral persistence at the genotype level, the kinetics of viral load, and/or likelihood of disease recurrence.

The methods, compositions, reagents, systems, and kits according to the present invention provide for an efficient test for initial testing or follow-up testing for genotyping and/or quantifying HPV types such as 16, 18, and/or 45. For example, methods include testing un-tested samples, verifying the presence of and/or quantifying HPV 16, 18, and/or 45 in positive samples, and verifying the absence of HPV 16, 18, and/or 45 in negative samples.

Because HPV viral loads have been correlated with disease progression in, for example, cervical cancer, HPV viral load can be a type-dependent risk marker for invasive carcinoma. Thus, methods include quantifying and categorizing viral load to diagnose risk of cancer and triage, including determining the priority of methods of treatment, etc.

Methods of the present invention also include screening samples for L1 deletions/mutations since E6 and E7 are in some cases targeted regions for determining whether a specific HPV type is present or absent.

The multiplex design of the present invention saves time and sample eluate when compared with typical individual testing/genotyping of HPV types such as 16, 18, and/or 45.

Primers and Probes

The primers and probes of the present invention are capable of targeting genes in the E6 and E7 regions for HPV genotypes 16, 18, and 45. The E6 and E7 regions are suitable target regions because they are not as prone to mutagenic events during integration as the L1 region is. The primers and probes are specific for HPV 16, 18, and 45 and do not exhibit any substantial cross-reactivity among themselves or among other HPV types. The primers and probes may be sensitive in detecting the presence of HPV 16, 18, or 45 with as few as 5 copies of target sequence per reaction present.

In embodiments, the primers may be from about 15 to 50 nucleotides in length, such as from about 18 to 30, about 20 to 26, or about 22 to 24 nucleotides. By way of example and not limitation, the primers may be not more than 50 nucleotides in length, not more than 30 nucleotides in length, not more than 26 nucleotides in length, or not more than 24 nucleotides in length. The probes may be from about 15 to about 60 nucleotides in length, such as from about 18 to 40, about 20 to 35, or about 22 to 30. By way of example and not limitation, the probes may be not more than 60 nucleotides in length, not more than 40 nucleotides in length, not more than 35 nucleotides in length, or not more than 30 nucleotides in length.

Exemplary primers and probes are shown in the tables below for each of HPV types 16, 18, and 45. For type 45, two primer and probe sets are shown with set No. 1 (SEQ ID NOs 7-9) being a preferred embodiment for multiplexing analysis. In the below tables, the probe sequences are conjugated with a signal on the 5′ end and a quencher on the 3′ end. These molecules are shown below (bold and underlined), but they are not part of the nucleic acid sequence represented by the respective SEQ ID NOs.

TABLE 1
HPV 16 Primer/Probe Set:

| | | |
|---|---|---|
| HPV 16 | SEQ ID | 5′ AGA ACC GGA CAG AGC CCA TTA CAA 3′ |
| primer A | NO. 1 | |
| HPV 16 | SEQ ID | 5′ GCA CAC AAT TCC TAG TGT GCC CAT 3′ |
| primer B | NO. 2 | |
| HPC 16 | SEQ ID | 5′ ROX-AC GCT TCG GTT GTG CGT ACA AAG CA-IAbRQSp 3′ |
| Probe | NO. 3 | |

TABLE 2
HPV 18 Primer/Probe Set:

| | | |
|---|---|---|
| HPV 18 | SEQ ID | 5′ TCA TCA ACA TTT ACC AGC CCG ACG 3′ |
| primer A | NO. 4 | |
| HPV 18 | SEQ ID | 5′ GAA ACA GCT GCT GGA ATG CTC GAA 3′ |
| primer B | NO. 5 | |
| HPC 18 | SEQ ID | 5′ HEX-AGC CGA ACC ACA ACG TCA CAC AAT GT-IABkFQ 3′ |
| Probe | NO. 6 | |

TABLE 3
HPV 45 Primer/Probe Set No. 1:

| | | |
|---|---|---|
| HPV 45 | SEQ ID | 5′ TTG ACG ATC CAA AGC AAC GAC CCT 3′ |
| primer A | NO. 7 | |
| HPV 45 | SEQ ID | 5′ CCT CTG TGC GTT CCA ATG TTG CTT 3′ |
| primer B | NO. 8 | |
| HPV 45 | SEQ ID | 5′ FAM-ACA AGC TAC CAG ATT TGT GCA CAG AAT TGA- |
| Probe 1 | NO. 9 | IABkFQ 3′ |

TABLE 4
HPV 45 Primer/Probe Set No. 2:

| | | |
|---|---|---|
| HPV 45 | SEQ ID | 5′ TTG TTA ATA AGG TGC CTG CGG TGC 3′ |
| primer C | NO. 10 | |
| HPV 45 | SEQ ID | 5′ TGT TTC CCT ACG TCT GCG AAG TCT 3′ |
| primer D | NO. 11 | |
| HPC 45 | SEQ ID | 5′ FAM-ATA CAT GTT GTG ACC AGG CAC GGC AA/31ABkFQ/ 3′ |
| Probe 2 | NO. 12 | |

In some embodiments a control may be used with the above primer and probes sets, for example, a beta-globin primer and probe set may be used with the multiplex system with a fourth detectable signal. Any suitable control may be used, so long as it does not interfere with the detection and quantification of the above exemplified primer and probe sets. The following primer and probe sets may be used as controls, with set No. 1 being preferred for multiplexing analysis.

TABLE 5
Beta-globin Primer/Probe Set No. 1:

| | | |
|---|---|---|
| Beta Globin | SEQ ID | 5′ GAA GAG CCA AGG ACA GGT AC 3′ |
| primer A | NO. 16 | |
| Beta Globin | SEQ ID | 5′ CAA CTT CAT CCA CGT TCA CC 3′ |
| primer B | NO. 17 | |
| Beta Globin | SEQ ID | 5′ CCC TAG GGT TGG CCA ATC TAC TC-IABkFQ 3′ |
| Probe 1 | NO. 18 | |

TABLE 6
Beta-globin Primer/Probe Set No. 2:

| Beta Globin | SEQ ID | 5′ ACA CAA CTG TGT TCA CTA GC 3′ |
|---|---|---|
| primer C | NO. 19 | |
| Beta Globin | SEQ ID | 5′ CAA CTT CAT CCA CGT TCA CC 3′ |
| primer D | NO. 20 | |
| Beta Globin | SEQ ID | 5′ TCA AAC AGA CAC CAT GGT GCA TCT GAC TCC-IABkFQ |
| Probe 2 | NO. 21 | 3 ′ |

The primers and probes may also include those primers and probes with about 70% to 100% identity and/or homology with the various primers and probes, such as 75% or more, 80% or more, 85% or more, 90% or more, 95% or more, and/or 98% or more identity and/or homology. By way of example and not limitation, the primer and/or probe may have from 70% to 100% identity and/or homology with a sequence selected from the group consisting of SEQ ID NO: 1 to SEQ ID NO: 12 and SEQ ID NO: 16 to SEQ ID NO: 21. As a further example, the probe may have 75% or more, 80% or more, 85% or more, 90% or more, 95% or more, and/or 98% or more identity and/or homology with a sequence selected from the group consisting of SEQ ID NO: 1 to SEQ ID NO: 12 and SEQ ID NO: 16 to SEQ ID NO: 21.

In an embodiment, a nucleic acid comprising a 3′ terminal sequence capable of hybridizing to nucleotides 695 to 719 of SEQ ID NO: 13 or the complement thereof is used as an HPV 16 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 695 to 719 of SEQ ID NO: 13 or the complement thereof. As another example, the HPV16 E6/E7-specific primer consists of a sequence having 70% or more, 75% or more, 80% or more, 85% or more, 90% or more, 95% or more, and/or 98% or more identity and/or homology to nucleotides 695 to 719 of SEQ ID NO: 13 or the complement thereof.

In another embodiment, a nucleic acid comprising a 3′ terminal sequence capable of hybridizing to nucleotides 811 to 834 of SEQ ID NO. 13 or a complement thereof is used an HPV 16 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 811 to 834 of SEQ ID NO: 13 or the complement thereof. As another example, the HPV16 E6/E7-specific primer consists of a sequence having 70% or more, 75% or more, 80% or more, 85% or more, 90% or more, 95% or more, and/or 98% or more identity and/or homology to nucleotides 811 to 834 of SEQ ID NO: 13 or the complement thereof.

In another embodiment, a probe capable of hybridizing to nucleotides 751 to 775 of SEQ ID NO. 13 or the complement thereof is used as a probe. By way of example and not limitation, the probe comprises or consists of a sequence having 70% or more, 75% or more, 80% or more, 85% or more, 90% or more, 95% or more, and/or 98% or more identity and/or homology to nucleotides 751 to 775 of SEQ ID NO. 13 or the complement thereof.

In another embodiment, an HPV 16 E6/E7-specific primer set is provided comprising: (a) a first primer comprising a 3′ terminal sequence capable of hybridizing to the complement of nucleotides 695 to 719 of SEQ ID NO: 13; and (b) a second primer comprising a 3′ terminal sequence capable of hybridizing to nucleotides 811 to 834 of SEQ ID NO. 13.

In another embodiment, an HPV 16 E6/E7-specific primer/probe set is provided comprising: (a) a first primer comprising a 3′ terminal sequence capable of hybridizing to the complement of nucleotides 695 to 719 of SEQ ID NO: 13; (b) a second primer comprising a 3′ terminal sequence capable of hybridizing to nucleotides 811 to 834 of SEQ ID NO. 13; and (c) a probe capable of hybridizing to nucleotides 751 to 775 of SEQ ID NO. 13 or the complement thereof.

In an embodiment, a nucleic acid comprising a 3′ terminal sequence capable of hybridizing to nucleotides 724 to 747 of SEQ ID NO: 14 or the complement thereof is used as an HPV 18 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 724 to 747 of SEQ ID NO: 14 or the complement thereof. As another example, the HPV18 E6/E7-specific primer consists of a sequence having 70% or more, 75% or more, 80% or more, 85% or more, 90% or more, 95% or more, and/or 98% or more identity and/or homology to nucleotides 724 to 747 of SEQ ID NO: 14 or the complement thereof.

In another embodiment, a nucleic acid comprising a 3′ terminal sequence capable of hybridizing to nucleotides 837 to 860 of SEQ ID NO. 14 or a complement thereof is used an HPV 18 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 837 to 860 of SEQ ID NO. 14 or the complement thereof. As another example, the HPV 18 E6/E7-specific primer consists of a sequence having 70% or more, 75% or more, 80% or more, 85% or more, 90% or more, 95% or more, and/or 98% or more identity and/or homology to nucleotides 837 to 860 of SEQ ID NO. 14 or the complement thereof.

In another embodiment, a probe capable of hybridizing to nucleotides 748 to 773 of SEQ ID NO. 14 or the complement thereof is used as a probe. By way of example and not limitation, the probe comprises or consists of a sequence having 70% or more, 75% or more, 80% or more, 85% or more, 90% or more, 95% or more, and/or 98% or more identity and/or homology to nucleotides 837 to 860 of SEQ ID NO. 14 or the complement thereof.

In another embodiment, an HPV 18 E6/E7-specific primer set is provided comprising: (a) a first primer comprising 3′ terminal sequence capable of hybridizing to the complement nucleotides 724 to 747 of SEQ ID NO: 14; and (b) a second primer comprising a 3′ terminal sequence capable of hybridizing to nucleotides 837 to 860 of SEQ ID NO. 14.

In another embodiment, an HPV 18 E6/E7-specific primer set is provided comprising: (a) a first primer comprising 3′ terminal sequence capable of hybridizing to the complement nucleotides 724 to 747 of SEQ ID NO: 14; (b) a second primer comprising a 3′ terminal sequence capable of hybridizing to nucleotides 837 to 860 of SEQ ID NO. 14; and (c) a probe capable of hybridizing to nucleotides 748 to 773 of SEQ ID NO. 14 or the complement thereof.

In an embodiment, a nucleic acid comprising a 3′ terminal sequence capable of hybridizing to nucleotides 112 to 135 of SEQ ID NO: 15 or the complement thereof is used as an HPV45 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 112 to 135 of SEQ ID NO: 15 or the complement thereof. As another example, the HPV45 E6/E7-specific primer consists of a sequence having 70% or more, 75% or more, 80% or more, 85% or more, 90% or more, 95% or more, and/or 98% or more identity and/or homology to nucleotides 112 to 135 of SEQ ID NO: 15 or the complement thereof.

In another embodiment, a nucleic acid comprising a 3′ terminal sequence capable of hybridizing to nucleotides 208 to 231 of SEQ ID NO. 15 or a complement thereof is used an HPV45 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 208 to 231 of SEQ ID NO. 15 or the complement thereof. As another example, the HPV45 E6/E7-specific primer consists of a sequence having 70% or more, 75% or more, 80% or more, 85% or more, 90% or more, 95% or more, and/or 98% or more identity and/or homology to nucleotides 208 to 231 of SEQ ID NO. 15 or the complement thereof.

In another embodiment, a probe capable of hybridizing to nucleotides 136 to 165 of SEQ ID NO. 15 or the complement thereof is used as a probe. By way of example and not limitation, the probe comprises or consists of a sequence having 70% or more, 75% or more, 80% or more, 85% or more, 90% or more, 95% or more, and/or 98% or more identity and/or homology to nucleotides 136 to 165 of SEQ ID NO. 15 or the complement thereof.

In another embodiment, an HPV 45 E6/E7-specific primer set is provided comprising: (a) a first primer comprising 3′ terminal sequence capable of hybridizing to the complement nucleotides 112 to 135 of SEQ ID NO: 15; and (b) a second primer comprising a 3′ terminal sequence capable of hybridizing to nucleotides 208 to 231 of SEQ ID NO. 15.

In another embodiment, an HPV 45 E6/E7-specific primer set is provided comprising: (a) a first primer comprising 3′ terminal sequence capable of hybridizing to the complement nucleotides 112 to 135 of SEQ ID NO: 15; (b) a second primer comprising a 3′ terminal sequence capable of hybridizing to nucleotides 208 to 231 of SEQ ID NO. 15; and (c) a probe capable of hybridizing to nucleotides 136 to 165 of SEQ ID NO. 15 or the complement thereof.

In an embodiment, a nucleic acid comprising a 3′ terminal sequence capable of hybridizing to nucleotides 402 to 425 of SEQ ID NO: 15 or the complement thereof is used as an HPV45 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 402 to 425 of SEQ ID NO: 15 or the complement thereof. As another example, the HPV45 E6/E7-specific primer consists of a sequence having 70% or more, 75% or more, 80% or more, 85% or more, 90% or more, 95% or more, and/or 98% or more identity and/or homology to nucleotides 402 to 425 of SEQ ID NO: 15 or the complement thereof.

In another embodiment, a nucleic acid comprising a 3′ terminal sequence capable of hybridizing to nucleotides 546 to 569 of SEQ ID NO. 15 or a complement thereof is used an HPV45 E6/E7-specific primer. By way of example and not limitation, the 3′ terminal sequence comprises or consists of a sequence having 70% or more, 75% or more, 80% or more, 85% or more, 90% or more, 95% or more, and/or 98% or more identity and/or homology to nucleotides 546 to 569 of SEQ ID NO. 15 or the complement thereof. As another example, the HPV45 E6/E7-specific primer consists of a sequence having 70% or more, 75% or more, 80% or more, 85% or more, 90% or more, 95% or more, and/or 98% or more identity and/or homology to nucleotides 546 to 569 of SEQ ID NO. 15 or the complement thereof.

In another embodiment, a probe capable of hybridizing to nucleotides 517 to 542 of SEQ ID NO. 15 or the complement thereof is used as a probe. By way of example and not limitation, the probe comprises or consists of a sequence having 70% or more, 75% or more, 80% or more, 85% or more, 90% or more, 95% or more, and/or 98% or more identity and/or homology to nucleotides 517 to 542 of SEQ ID NO. 15 or the complement thereof.

In another embodiment, an HPV 45 E6/E7-specific primer set is provided comprising: (a) a first primer comprising 3′ terminal sequence capable of hybridizing to the complement nucleotides 402 to 425 of SEQ ID NO: 15; and (b) a second primer comprising a 3′ terminal sequence capable of hybridizing to nucleotides 546 to 569 of SEQ ID NO. 15.

In another embodiment, an HPV 45 E6/E7-specific primer set is provided comprising: (a) a first primer comprising 3′ terminal sequence capable of hybridizing to the complement nucleotides 402 to 425 of SEQ ID NO: 15; (b) a second primer comprising a 3′ terminal sequence capable of hybridizing to nucleotides 546 to 569 of SEQ ID NO. 15; and (c) a probe capable of hybridizing to nucleotides 517 to 542 of SEQ ID NO. 15 or the complement thereof.

As used herein, the phrases “HPV 16 E6/E7-specific primer,” “HPV 18 E6/E7-specific primer,” and “HPV 45 E6/E7-specific primer” refer to nucleic acid primers capable of mediating the amplification of a portion of the E6/E7 region of the genome of the indicated HPV type, but which is not capable of mediating the amplification of a portion of the E6/E7 region of the genome of a different HPV type.

In one embodiment, the probes are designed to include a detectable signal, such as a radioactive, biotin, or fluorescent signal, suitable for the independent detection of the genotypes to be tested. Any suitable detectable signal may be used, for example, HPV 16 may be labeled with ROX, an orange fluorescent molecule; HPV 18 may be labeled with HEX, a yellow fluorescent molecule, HPV 45 may be labeled with FAM, a green fluorescent molecule, and the control, beta-globin, may be labeled with Cy5, a red fluorescent molecule. In embodiments, as shown in the tables above, the probes may be TaqMan probes with a fluorophore covalently attached to the 5′ end of the probe and a quencher attached at the 3′ end of the probe. When the fluorophore and quencher are in proximity, any fluorescence is inhibited by the quencher. However, when the Taq polymerase extends the primer, the probe is degraded by the exonuclease activity of the polymerase, thereby freeing the fluorophore from the quenching activity of the quencher. Any suitable combination of label and quencher may be used.

In another embodiment, the probe is utilized to generate a DNA:RNA hybrid for use in hybrid capture. Hybrid capture technology utilizes certain antibodies capable of binding to DNA:RNA hybrids in various methods of purifying and detecting specific target nucleic acids in a sample. Various iterations of the hybrid capture method are described in, inter alia, U.S. Pat. Nos. 5,994,079, 6,027,897, 6,277,579, 6,686,151, and 7,439,016; US Patent Publication Nos. 2006/0051809 A1, 2009/0162851 A1, and 2009-0298187 A1; and PCT Publication No. WO 01/96608, each of which is incorporated herein by reference in its entirety. The basic hybrid capture protocol comprises: (1) hybridizing a nucleic acid probe to the target nucleic acid to generate a DNA:RNA hybrid; (2) associating the DNA:RNA hybrid with a solid phase to facilitate isolation of the target nucleic acid; and (3) detecting the DNA:RNA hybrid. In various iterations, anti-DNA:RNA hybrid antibodies can be used in either step (2) or step (3). By way of example and not limitation, the anti-DNA:RNA hybrid antibody may be bound to the solid phase (covalently or otherwise), thereby mediating “capture” of the DNA:RNA hybrid to the solid phase. Alternatively, a nucleic acid probe bound to the solid phase (covalently or otherwise) may capture the DNA:RNA hybrid to the solid phase, which may then be detected by a detectably labeled anti-DNA:RNA hybrid antibody.

Specimen Preparation

Any suitable specimen preparation method may be used. When the methods are used to verify or quantify a positive or negative result, a sample of the already purified specimen may be used. When the methods are used to make an initial determination/quantification of HPV 16, 18, and/or 45, specimens may be purified using any suitable sample purification method.

PCR Equipment and Conditions

Any suitable PCR equipment and conditions capable of real-time PCR analysis may be used to amplify and detect the target PCR product. In embodiments, a Rotor-Gene Q, made by Qiagen, may be used.

### EXAMPLES

An HPV 16 Primer/Probe Set (SEQ ID NOs. 1-3), HPV 18 Primer/Probe Set (SEQ ID NOs. 4-6), and HPV 45 Primer Probe Set No. 1 (SEQ ID NOs. 7-9) were evaluated in singleplex real time PCR. Analytical specificity against both high risk (HR) and low risk (LR) HPV types was tested using plasmids in a clean system at 1E+7 copies/assay. HPV types 6, 11, 16, 18, 26, 31, 33, 35, 39, 40, 42, 43, 45, 51, 52, 53, 56, 58, 59, 61, 66, 67, 68, 69, 71, 72, 73, 81, 82, 2, 3, 13, 30, 34, 44, 70, and 83 were tested. The results showed that the Primer/Probe sets were specific, respectively, for HPV 16, 18, and 45.

The Primer/Probe sets were also tested against possible interfering substances, namely, blood, contraceptive jelly, spermicide, moisturizer, hemorrhoidal anesthetic, body oil, anti-fungal cream, vaginal lubricants, and feminine spray. The results showed that these substances did not interfere with the specificity of the Primer/Probe sets.

The Primer/Probe sets were also tested against 16 non-HPV pathogens, per FDA recommendations.

The HPV 16 Primer/Probe Set, HPV 18 Primer/Probe Set, and HPV 45 Primer Probe Set No. 1 were verified by BLAST analysis. The sequences were compared to all relevant mucosal HPV types and all known subtypes of HPV 16, 18, and 45 in the NCBI database. No known subtypes of HPV 16, 18, and 45 produced greater than 10% mismatches. All known subtypes had a greater than 90% homology. Sequences with greater than 80% homology to the HPV 16, 18, and 45 primers and probes are shown in Table 7.

TABLE 7
Sequences With Greater Than 80% Homology to HPV 16, 18,
and 45 Primers and Probes

| Sequence | HPV Type | BLAST (HPV Type) | % Identity |
|---|---|---|---|
| Forward Primer | 16 | 31 | 86.36 |
| Forward Primer | 18 | 97 | 87.5 |
| Forward Primer | 18 | 45 | 83.33 |
| Forward Primer | 45 | 59 | 81.82 |
| Forward Primer | 45 | 37 | 80.95 |
| Reverse Primer | 16 | 73 | 91.67 |
| Reverse Primer | 16 | 35 | 87.5 |
| Reverse Primer | 16 | 67 | 86.96 |
| Reverse Primer | 16 | 43 | 86.96 |
| Reverse Primer | 16 | 32 | 86.36 |
| Reverse Primer | 16 | 34 | 83.33 |
| Reverse Primer | 16 | 7 | 83.33 |
| Reverse Primer | 16 | 11 | 86.36 |
| Reverse Primer | 16 | 42 | 86.36 |
| Reverse Primer | 16 | 71 | 81.82 |
| Reverse Primer | 16 | 91 | 81.82 |
| Reverse Primer | 18 | 59 | 90.48 |
| Reverse Primer | 18 | 45 | 82.61 |
| Reverse Primer | 18 | 67 | 80.95 |
| Reverse Primer | 45 | 97 | 91.67 |
| Reverse Primer | 45 | 110 | 85.71 |
| Probe | 18 | 45 | 91.67 |
| Probe | 18 | 97 | 91.67 |
| Probe | 18 | 68 | 90.48 |
| Probe | 18 | 80 | 86.36 |
| Probe | 18 | 113 | 86.36 |
| Probe | 45 | 18 | 86.67 |
| Probe | 45 | 97 | 86.67 |
| Probe | 45 | 16 | 91.3 |

Sample Preparation

Samples were purified using a modified QIAamp Media MDx Kit, which is a commercially available nucleic acid extraction and purification technology based on silica gel absorption in a column format. The modified steps from the standard protocol depending on the specimen type is shown below in Table 8.

TABLE 8
Modified Protocol Depending on Specimen Type

| | Input Sample | Modified Steps from | Elution |
|---|---|---|---|
| Specimen Type | Volume | Standard Protocol | Volume |
| Sample | 100 μL or 150 μL | Denatured specimen→ | 100 μL |
| Transport | denatured | Add 50 μL MES | |
| Media (STM) | | neutralization buffer | |
| PreservCyt ® | 250 μL+ | Centrifuge→decant→ | 100 μL |
| | | add 200 μL STM | |
| SurePath ™ | Pre-gradient or | Centrifuge→decant→ | 100 μL |
| | diluted sample: | add 200 μL 100 mM Tris- | |
| | 500 μL; post- | buffer pH 8 (or 9)→ | |
| | gradient (from | proteinase K incubation | |
| | total sample | was extended to one hour | |
| | volume @ 3 mL): | at 56° C. with shaking | |
| | 300 μL | at 600 RPM | |

Real-Time PCR Method

The purified samples were then used with the HPV 16 Primer/Probe Set, HPV 18 Primer/Probe Set, and HPV 45 Primer Probe Set No. 1 along with the beta-globin probe/primer Set No. 1. The mixture and conditions of the PCR method are shown below in Table 9.

TABLE 9
PCR Mixture and Cycling Conditions
PCR Mix

| | | |
|---|---|---|
| Total Reaction Volume | 25 | uL |
| Template | 5 | uL |
| HPV Primers | 200 | nM |
| HPV Probes | 200 | nM |
| Beta-globin IC Primers | 60 | nM |
| Beta-globin IC Probes | 60 | nM |
| QIAGEN QT Virus Master Mix (5X) | 1X | |

Rotor-Gene Q Conditions

| | | |
|---|---|---|
| Denaturation | 95° | C. for 5 minutes |
| Annealing | 95° | C. for 15 seconds |
| Extension | 60° | C. for 75 seconds |
| Cycles | 45 | |

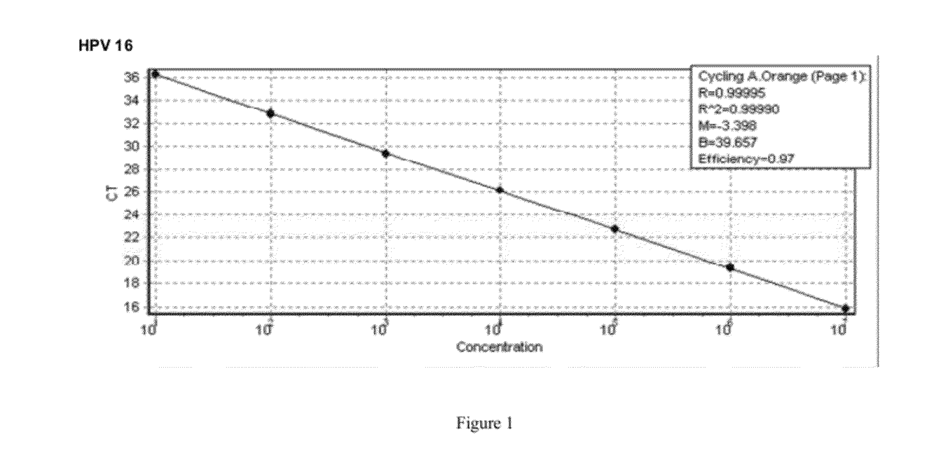

Assay Sensitivity: The dynamic range of each HPV 16, 18, and 45 multiplex primer/probe set was determined using a plasmid model. The dynamic range was determined to be at least 6 magnitudes. As illustrated in FIG. 1, a correlation coefficient (R2 value) of ≧0.97 and PCR efficiency of ≧0.97 was consistently achieved for HPV 16, 18, and 45 from 10⁷ to 10-genome copies/assay. Ct values and LOD for each HPV primer/probe were found to be comparable between singleplex assay detection and multiplex assay detection.

Assay Specificity: Each primer/probe pair was tested in multiplex for specificity against 27 HR and LR HPV types at 1×10⁷ copies/assay of each HPV type plasmid. The results are shown below in Tables 10 and 11.

TABLE 10
Assay Specificity for HR HPV Types

| | HPV Probe Type | | |
|---|---|---|---|
| HR HPV Target | 16 | 18 | 45 |
| 16 | pos | neg | neg |
| 18 | neg | pos | neg |
| 26 | neg | neg | neg |
| 31 | neg | neg | neg |
| 33 | neg | neg | neg |
| 35 | neg | neg | neg |
| 39 | neg | neg | neg |
| 45 | neg | *neg | pos |
| 51 | neg | neg | neg |
| 52 | neg | neg | neg |
| 56 | neg | neg | neg |
| 58 | neg | neg | neg |
| 59 | neg | neg | neg |
| 66 | neg | neg | neg |
| 68 | neg | neg | neg |
| 71 | neg | neg | neg |
| 72 | neg | neg | neg |
| 73 | neg | neg | neg |
| 82 | neg | neg | neg |

*HPV 18 primers and probes inconsistently detected HPV 45 target input of 10⁷ with Ct value of ≧40 and detection of ≧5 copies/assay.

TABLE 11
Assay Specificity for LR HPV Types

| | HPV Probe Type | | |
|---|---|---|---|
| LR HPV Target | 16 | 18 | 45 |
| 6 | neg | neg | neg |
| 11 | neg | neg | neg |
| 40 | neg | neg | neg |
| 42 | neg | neg | neg |
| 43 | neg | neg | neg |
| 2 | neg | neg | pos |
| 3 | neg | neg | neg |
| 13 | neg | neg | neg |
| 44 | neg | neg | neg |
| 53 | neg | neg | neg |
| 67 | neg | neg | neg |
| 69 | neg | neg | neg |
| 70 | neg | neg | neg |
| 83 | neg | neg | neg |
| 30 | neg | neg | neg |
| 34 | neg | neg | neg |
| 61 | neg | neg | neg |
| 81 | neg | neg | neg |

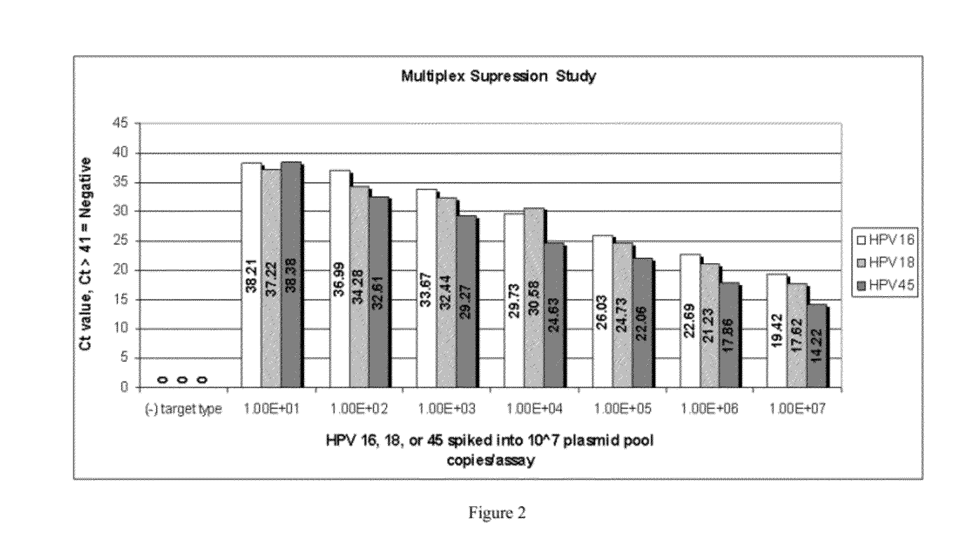

Assay Specificity-Suppression/Inhibition in Multiplex: To further test the specificity of the multiplex assay, HPV 16, 18, and 45 were diluted 10 fold from 10 copies to 10⁷ copies. These dilutions were tested in a plasmid pool of either HPV 16, 18, or 45+ HPV 39, 43, 67, and 83 at 10⁷ copies/assay of each HPV type. As illustrated in FIG. 2, suppression of the assays specificity to each of the target HPV types was minimal.

Clinical Sample Performance: Over 1000 cervical specimens HC2 screened and GP PCR/LMX genotyped from STM, PC, and SP media were tested in the multiplex assay format. The GP PCR Results are shown below in Tables 12-14.

TABLE 12
STM Specimen Results for GP PCR

| Specimen | HR (+) | LR (+) |
|---|---|---|
| STM8517 | 16 | 40 |
| STM8545 | 16, 52 | neg |
| STM8585 | 16, 18 | neg |
| STM8697 | 16, 68 | neg |
| STM8643 | 16, 31 | 13.32, 69, 86, 89, 90, 6 |
| STM16 | 16 | 10, 40 |
| STM112 | 16, 45 | 40, 86, 87 |
| STM8215 | 16 | neg |
| STM8790 | 16 | 81 |
| STM8705 | 16 | neg |
| STM8525 | 45 | 40 |
| STM8550 | 31 | 90 |

TABLE 13
PC Specimen Results for GP PCR

| Specimen | HR (+) | LR (+) |
|---|---|---|
| PC5635 | 16, 31 | 74, 90 |
| PC56181 | 16 | 70 |
| PC56360 | 16, 39 | 30, 72 |
| PC56603 | 16 | neg |
| PC56209 | 16, 39 | neg |
| PC56567 | 18, 16 | 40, 62, 74, 81 |
| PC56434 | 16 | 10, 13, 32, 62, 90 |
| PC56533 | 16, 68 | neg |
| PC53802 | 16 | 40 |
| PC54075 | 16, 18 | 67, 89 |
| PC54076 | 16 | 89, 6 |

TABLE 14
SP Specimen Results for GP PCR

| | | |
|---|---|---|
| SP10462 | 16 | neg |
| SP10556 | 16, 52 | neg |
| SP10583 | 16, 82 | neg |
| SP10596 | 16 | neg |
| SP10505 | 16, 66 | neg |
| SP10605 | 16 | neg |
| SP10472 | 16 | neg |
| SP10481 | 16, 18 | neg |
| SP10606 | 16 | 6 |
| SP10693 | 16 | 42 |
| SP10704 | 16, 51, 56 | neg |

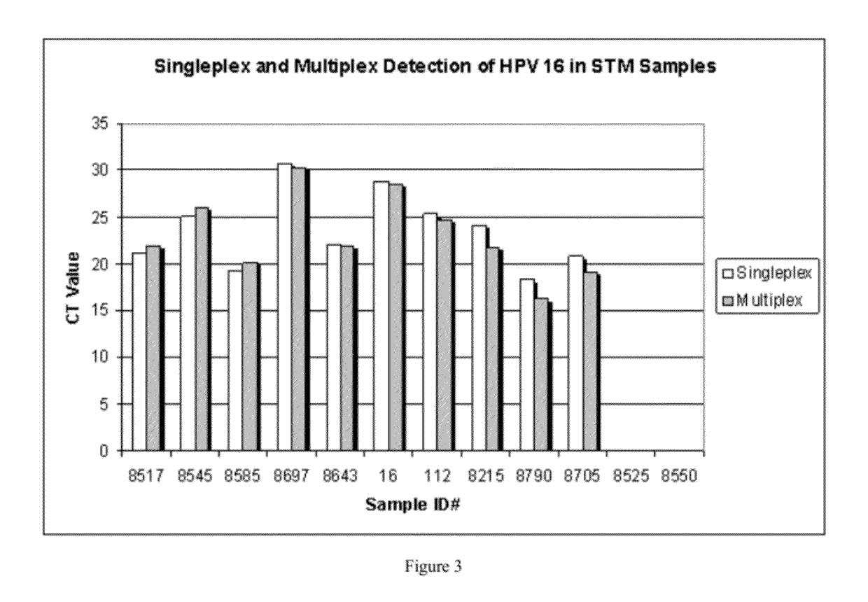

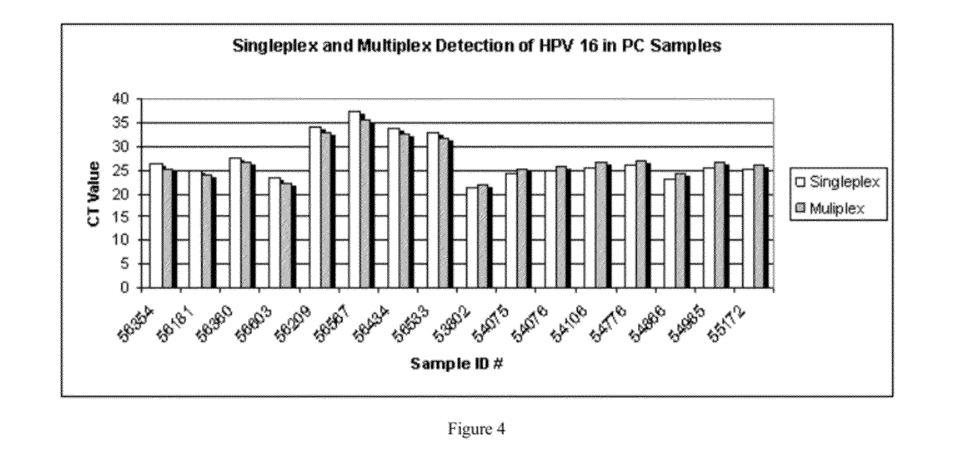

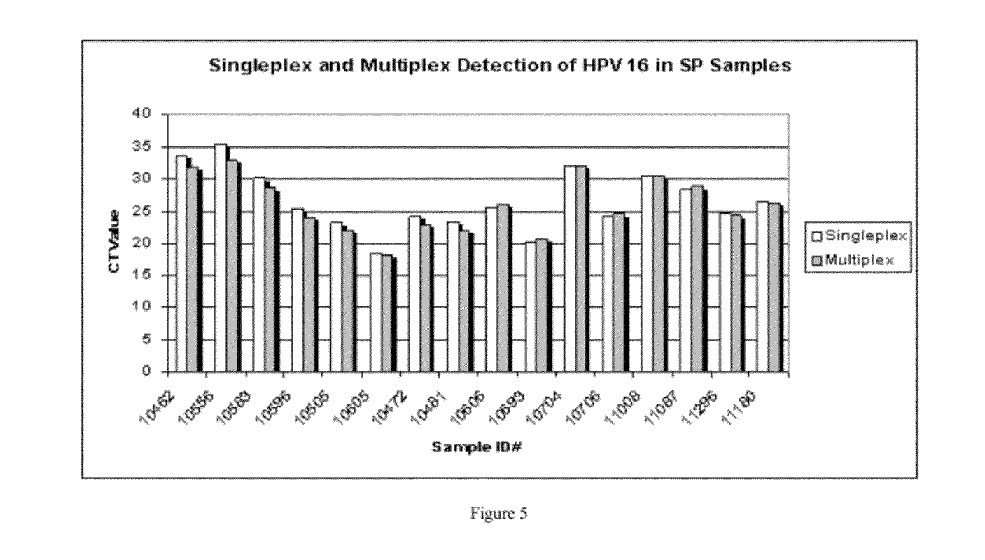

FIGS. 3-5 illustrate several examples of HPV 16 detection in single and multiplex assays for various sample types.

About 500 samples have been tested in both multiplex and singleplex. These tests showed comparable results in genotyping specimens positive or negative for the target HPV types (16, 18, and 45).

Clinical samples with multiple HPV infections were genotyped using GP 5+/6+ LMX Genotyping, as described in, for example, U.S. Pat. No. 6,352,825, the entire disclosure of which is herein incorporated by reference. Using about 254, of sample, the primers and probe sets for HPV 16, 18, and 45 were used to detect the presence or absence of HPV 16, 18, and 45. The results are summarized in Table 15 below.

TABLE 15
Examples of Clinical Samples with Multiple Infections

| | | GP 5+/6+ LMX Genotyping | | | | qPCR, copies/~25 uL Reaction | | |
|---|---|---|---|---|---|---|---|---|
| Plate Source | Media Type | Sample # | HR 1 | HR 2 | HR 3 | HPV 16 | HPV 18 | HPV 45 |
| 112409AM careHPV | DCM | 5567 | 16 | 18 | 39 | 1.21E+05 | 1.70E+05 | |
| Plate1 | | | | | | | | |
| 101909AM DCM | DCM | 229 | 16 | 45 | | 1.01E+05 | | 6.31E+04 |
| Samples | | | | | | | | |
| 101909AM DCM | DCM | 239 | 16 | 18 | 56 | 3.89E+04 | 6.20E+03 | |
| Samples | | | | | | | | |
| 101909AM DCM | DCM | 313 | 16 | 45 | | 3.70E+03 | | 1.00E+02 |
| Samples | | | | | | | | |
| 101909AM DCM | DCM | 373 | 18 | 39 | 45 | | 1.50E+02 | 2.50E+01 |
| Samples | | | | | | | | |
| 91109AM Plate 2 | DCM | 338 | 16 | 18 | 51 | 2.11E+04 | 2.66E+04 | |
| 010710AM ShubingPC | PC | 11253 | 16 | 18 | | 2.45E+02 | 2.06E+03 | |
| 113009AM Plate 1 | PC | 25136 | 16 | 18 | | 1.50E+01 | 3.50E+01 | |
| 110409AM Plate 1 | SP | 6502 | 18 | 35 | 45 | | 1.80E+03 | 2.99E+05 |
| Surepath | | | | | | | | |
| 011210AM PL1 Haiti | STM | 5567 | 16 | 18 | 39 | 1.08E+04 | 6.28E+03 | |
| STM | | | | | | | | |
| 011410AM PL2 Haiti | STM | 5941 | 16 | 18 | | 3.10E+03 | 4.20E+01 | |
| STM | | | | | | | | |
| 011410AM PL2 Haiti | STM | 6615 | 16 | 18 | 52 | 7.50E+01 | 2.70E+01 | |
| STM | | | | | | | | |
| 011210AM PL1 Haiti | STM | 6710 | 16 | 45 | | 2.40E+04 | | 1.26E+04 |
| STM | | | | | | | | |
| 042610AM Haiti STM | STM | 7336 | 16 | 45 | | 3.41E+02 | | 4.23E+03 |
| Morelia | PC | 1-0138 | 16 | 45 | | 1.22E+04 | | 2.25E+03 |

4-Plex Assays: Primers and Probes for Beta-Globin were used as an internal control along with the primer/probe sets for HPV 16, 18, and 45. Various samples were run with the 4-plex primers and probes. The samples are summarized below in Table 16.

TABLE 16
Samples in Run With 4-Plex To Test Specificity
SAMPLE

No template control (NTC) = Water
HPV 16 plasmid at 10³ c/rxn and 10⁵ c/rxn
HPV 18 plasmid at 10³ c/rxn and 10⁵ c/rxn
HPV 45 plasmid at 10³ c/rxn and 10⁵ c/rxn
Human Genomic DNA at 52 ng/rxn and 525 ng/rxn
Negative Clinical Pool
Negative Clinical Pool + HPV 16 plasmid spike of 10³ copies/rxn
Negative Clinical Pool + HPV 18 plasmid spike of 10³ copies/rxn
Negative Clinical Pool + HPV 45 plasmid spike of 10³ copies/rxn

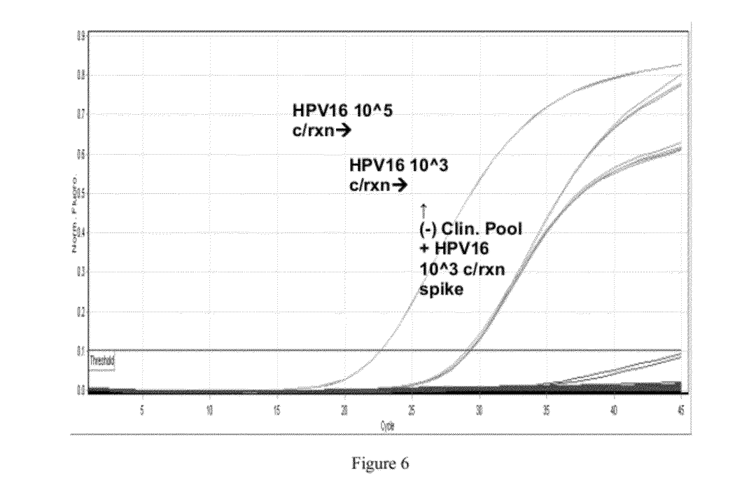

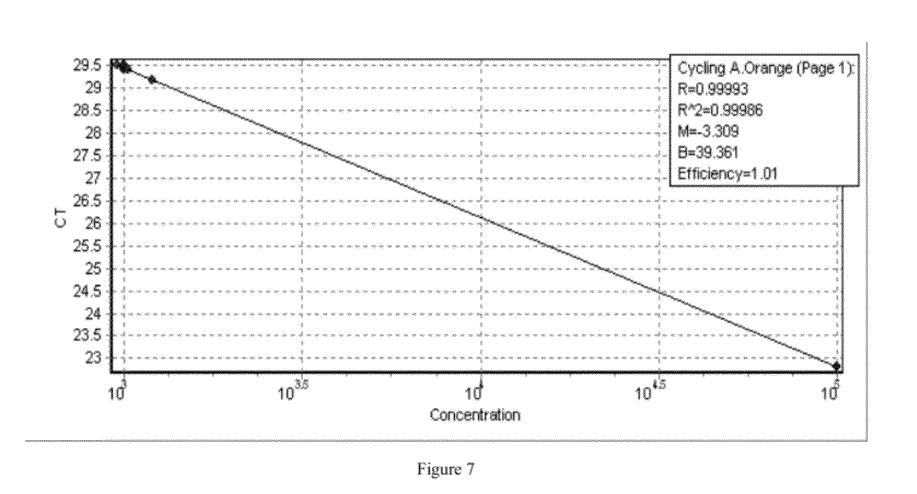

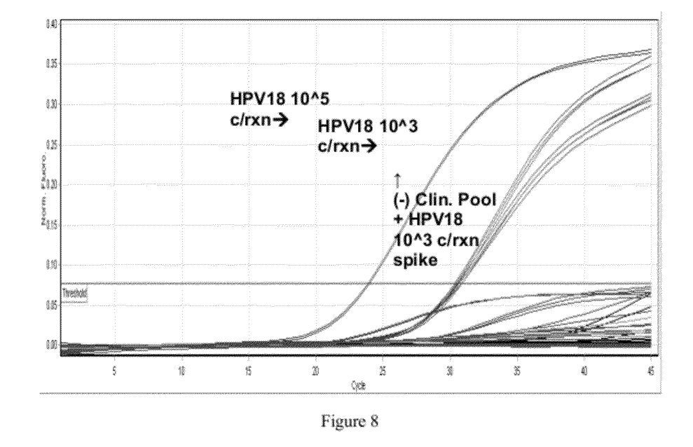

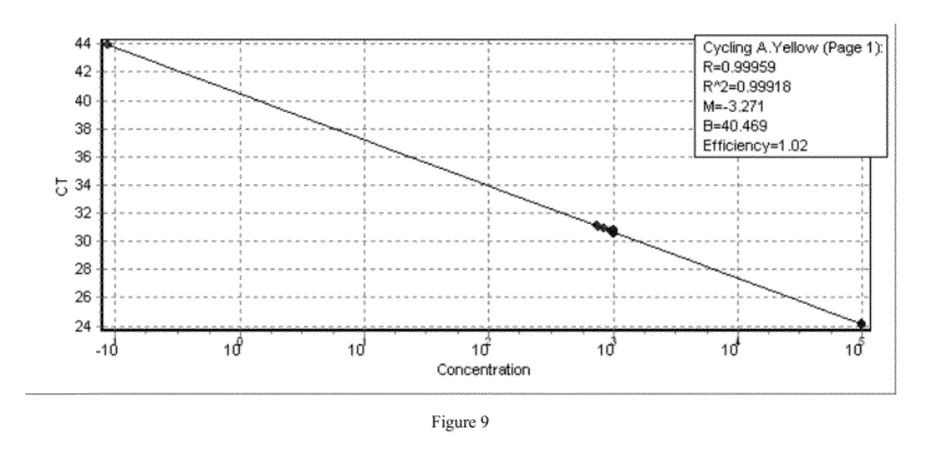

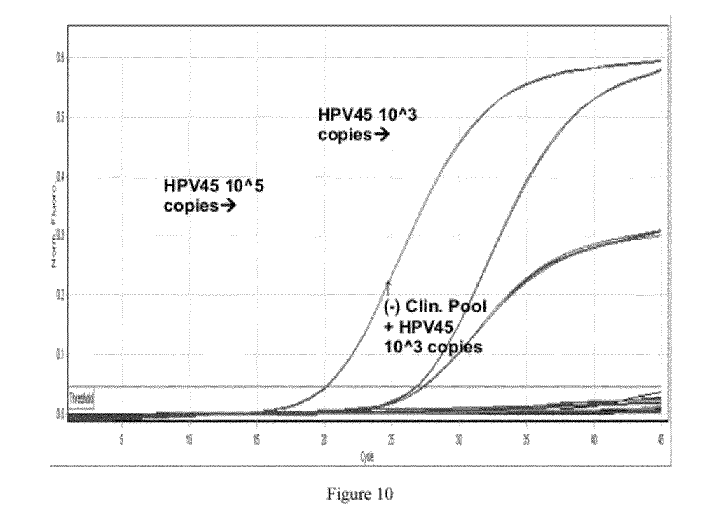

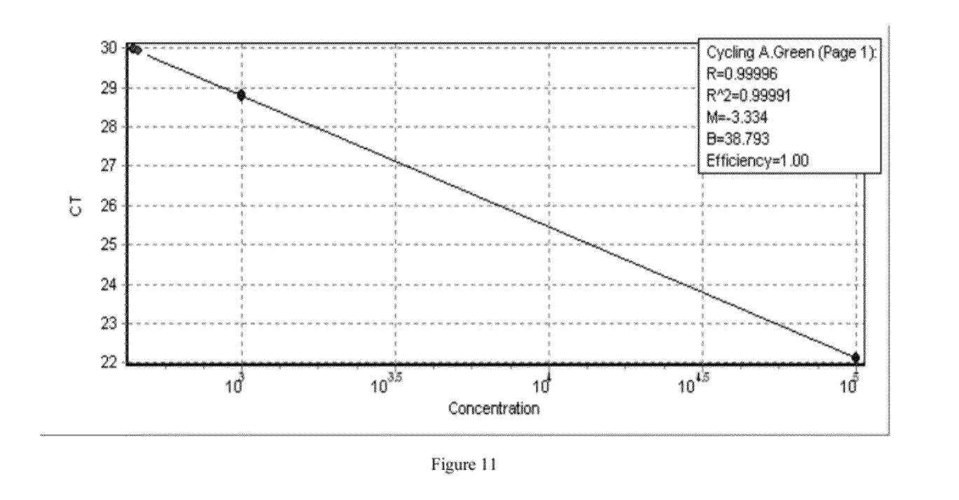

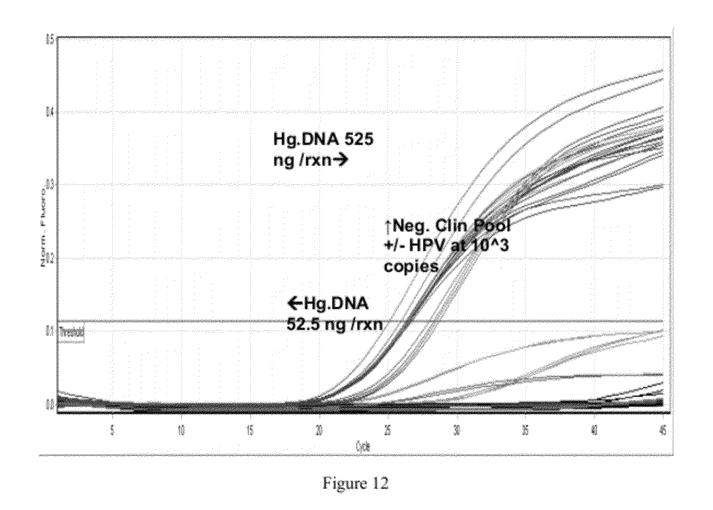

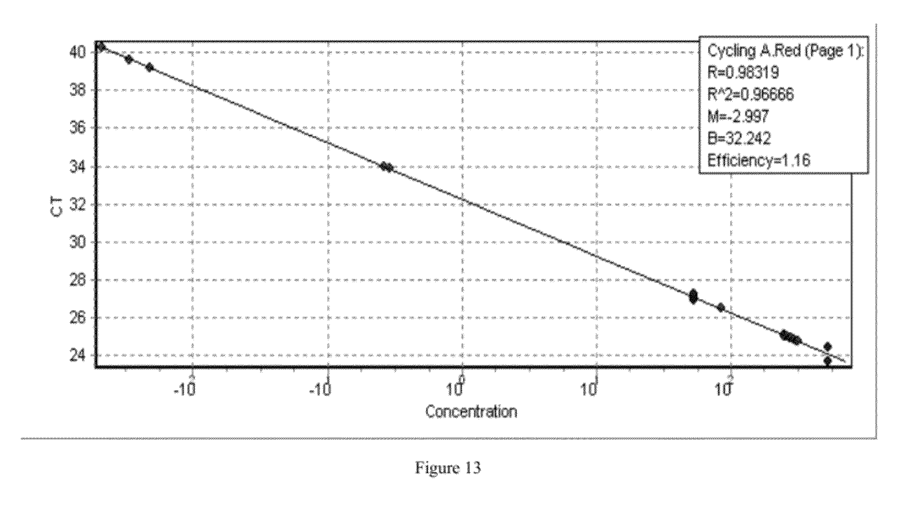

The results of running these samples are illustrated in FIGS. 6-13. The assays demonstrated that:
the orange channel is specific only to samples containing HPV 16 DNA detected by the ROX probe;
the yellow channel is specific only to samples containing HPV 18 DNA detected by the HEX probe;
the green channel is specific only to samples containing HPV 45 DNA detected by the FAM probe;
the red channel is specific only to negative clinical background +/−HPV 16, 18, or 45 plasmid detected by the Cy5 probe.
   Thus, the specificity of each channel to the selected fluorophore in the multiplex format including the internal control primers and probes were demonstrated.

In addition to specificity, the 4-Plex assay demonstrates sensitivity in detecting the presence of HPV 16, 18, and/or 45. The following samples listed in Table 17 were run with the 4-plex primers and probes.

TABLE 17
Samples in Run With 4-Plex To Test Sensitivity
SAMPLES

NTC = Water
HPV 16 plasmid at 5, 10, 100, 10³ c/rxn
HPV 18 plasmid at 5, 10, 100, 10³ c/rxn
HPV 45 plasmid at 5, 10, 100, 10³ c/rxn

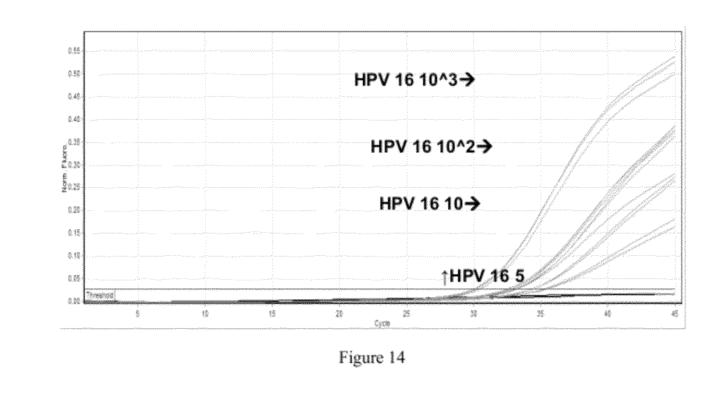

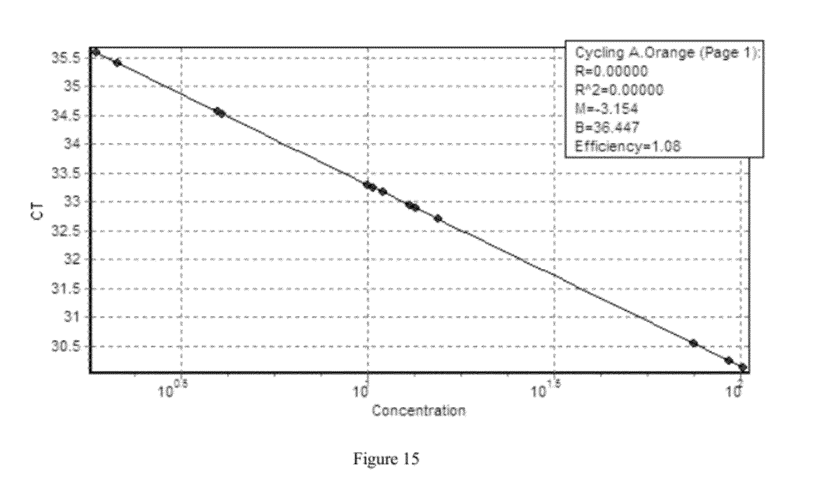

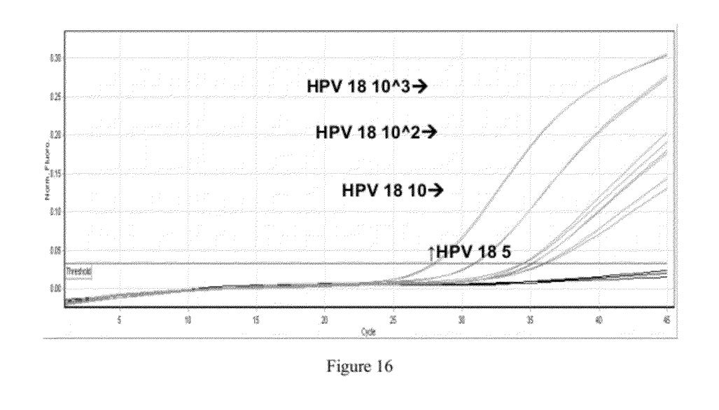

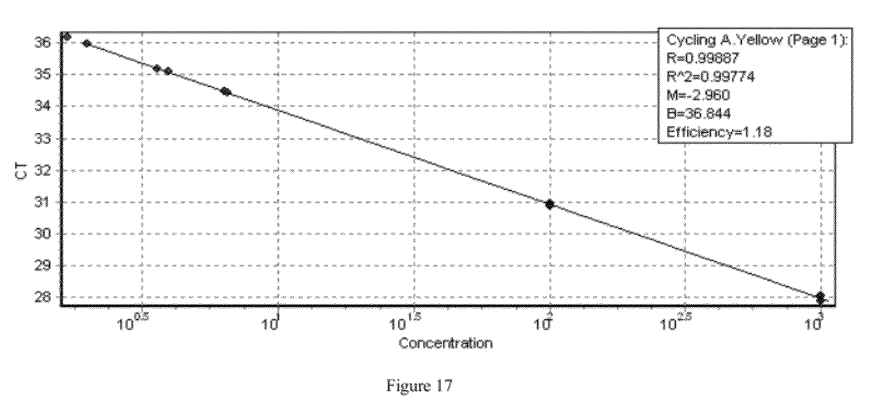

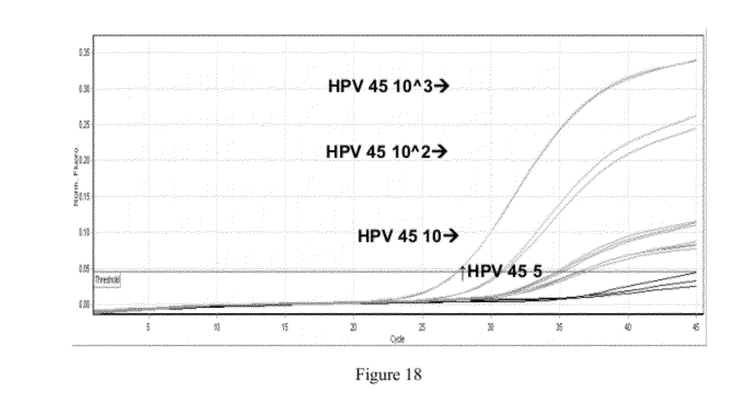

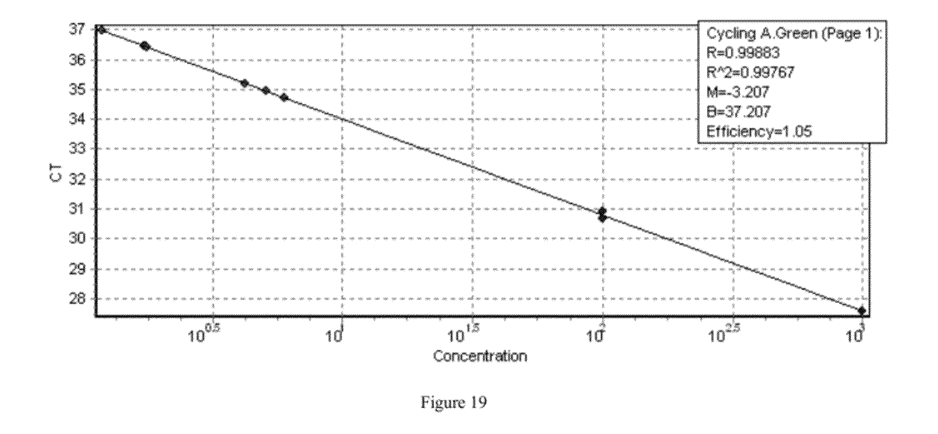

Each of the sample dilutions were spiked into a negative clinical eluate pool. The results of running these samples are illustrated in FIGS. 14-19. Each of the primer/probe sets demonstrated sensitivity, being detectable at levels of at least 5 copies/reaction in the presence of HPV negative clinical background.

Sequencing Samples:

Of the numerous samples tested above, 34 samples (17 multiplex positive and 17 multiplex negative) were sequenced. All the sample sequencing matched with the multiplex data, thus providing further verification of the specificity, sensitivity, and accuracy of the multiplex assay.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A nucleic acid primer or probe specific for a target sequence in the E6/E7 region of a high-risk HPV genome, wherein:
a. said nucleic acid primer or probe has 80% identity or greater across its entire length to both the target sequence and at least one nucleic acid sequence in E6/E7 region of a genome of a subtype thereof; and
b. said nucleic acid primer or probe does not hybridize to a nucleic acid derived from a different HPV type.

2. The nucleic acid primer or probe of claim 1, wherein the high risk HPV genome is selected from the group consisting of HPV 16, HPV 18, and HPV 45.

3. The nucleic acid primer or probe of claim 2 comprising a sequence having about 70% to 100% identity with a sequence selected from the group consisting of SEQ ID NO:1 to SEQ ID NO:12 and SEQ ID NO:16 to SEQ ID NO:21, or a complement thereof.

4. The nucleic acid primer or probe of claim 1 consisting of a sequence selected from the group consisting of SEQ ID NO:1 to SEQ ID NO:12 and SEQ ID NO:16 to SEQ ID NO:21, and/or a complement thereof.

5. The nucleic acid probe of claim 3 comprising a sequence having about 70% to 100% identity with a sequence selected from the group consisting of SEQ ID NO:3, SEQ ID NO:6, SEQ ID NO:9, SEQ ID NO:12, SEQ ID NO:18, and SEQ ID NO:21, or a complement thereof.

6. The nucleic acid primer of claim 3, wherein the primer is capable of amplifying a portion of the E6/E7 region of the HPV 16 genome, but not an E6/E7 region of HPV 18 or HPV 45 genome.

7. The nucleic acid primer of claim 6 comprising SEQ ID NO:1 or SEQ ID NO:2 or a complement thereof.

8. The nucleic acid primer of claim 3, wherein the primer is capable of amplifying a portion of the E6/E7 region of the HPV 18 genome, but not an E6/E7 region of HPV 16 or HPV 45 genome.

9. The nucleic acid primer of claim 8 comprising SEQ ID NO:4 or SEQ ID NO:5.

10. The nucleic acid primer of claim 3, wherein the primer is capable of amplifying a portion of the E6/E7 region of the HPV 45 genome, but not an E6/E7 region of an HPV 16 genome or HPV 18 genome

11. The nucleic acid primer of claim 10 comprising SEQ ID NO:7 or SEQ ID NO:8.

12. A primer set, comprising at least one primer according to claim 3.

13. The primer set of claim 12 comprising at least one primer pair selected from the group consisting of:
a. a primer pair capable of amplifying a portion of the E6/E7 region of the HPV 16 genome, but not an E6/E7 region of HPV 18 or HPV 45 genome;
b. a primer pair capable of amplifying a portion of the E6/E7 region of the HPV 18 genome, but not an E6/E7 region of HPV 16 or HPV 45 genome; and
c. a primer pair capable of amplifying a portion of the E6/E7 region of the HPV 45 genome, but not an E6/E7 region of HPV 16 or HPV 18 genome.

14. The primer set according to claim 13 comprising a primer pair selected from the group consisting of:
a. a first primer having about 70% to 100% identity with SEQ ID NO: 1 and a second primer having about 70% to 100% identity SEQ ID NO: 2,
b. a first primer having about 70% to 100% identity with SEQ ID NO: 4 and a second primer having about 70% to 100% identity SEQ ID NO: 5,
c. a first primer having about 70% to 100% identity with SEQ ID NO: 7 and a second primer having about 70% to 100% identity SEQ ID NO: 8,
d. a first primer having about 70% to 100% identity with SEQ ID NO: 10 and a second primer having about 70% to 100% identity SEQ ID NO: 11,
e. a first primer having about 70% to 100% identity with SEQ ID NO: 16 and a second primer having about 70% to 100% identity SEQ ID NO: 17,
f. a first primer having about 70% to 100% identity with SEQ ID NO: 19 and a second primer having about 70% to 100% identity SEQ ID NO: 20.

15. The primer set according to claim 13 comprising a primer pair selected from the group consisting of:
a. a first primer consisting of SEQ ID NO: 1 and a second primer having about 70% to 100% identity SEQ ID NO: 2,
b. a first primer consisting of SEQ ID NO: 4 and a second primer having about 70% to 100% identity SEQ ID NO: 5,
c. a first primer consisting of SEQ ID NO: 7 and a second primer having about 70% to 100% identity SEQ ID NO: 8,
d. a first primer consisting of SEQ ID NO: 10 and a second primer having about 70% to 100% identity SEQ ID NO: 11,
e. a first primer consisting of SEQ ID NO: 16 and a second primer having about 70% to 100% identity SEQ ID NO: 17,
f. a first primer consisting of SEQ ID NO: 19 and a second primer having about 70% to 100% identity SEQ ID NO: 20.

16. A kit for the detection of HPV 16, 18, and/or 45, said kit comprising a nucleic acid primer or probe according to claim 1.

17. The kit of claim 15 comprising at least a nucleic acid primer and at least one nucleic acid probe, wherein:
a. said nucleic acid primer comprises a sequence having about 70% to 100% identity with a sequence selected from the group consisting of SEQ ID NO:1, SEQ ID NO:2, SEQ ID NO:4, SEQ ID NO:5, SEQ ID NO:7, SEQ ID NO:8, SEQ ID NO:10, SEQ ID NO:11, SEQ ID NO:16, SEQ ID NO:17, SEQ ID NO:19, and SEQ ID NO:20, or a complement thereof; and
b. said nucleic acid probe comprises a sequence having about 70% to 100% identity with a sequence selected from the group consisting of SEQ ID NO:3, SEQ ID NO:6, SEQ ID NO:9, SEQ ID NO:12, SEQ ID NO:18, and SEQ ID NO:21, or a complement thereof.

18. The kit according to claim 17 comprising an HPV 16 primer and probe set, an HPV 18 primer and probe set, and/or an HPV 45 primer and probe set.

19. The kit according to claim 18, wherein:
a. the HPV 16 primer and probe set comprises:
(i) a primer having 70% to 100% identity with a sequence of SEQ ID NO:1,
(ii) a primer having 70% to 100% identity with a sequence of SEQ ID NO:2, and,
(iii) a probe having 70% to 100% identity with a sequence of SEQ ID NO:3;
b. the HPV 18 primer and probe set comprises:
(i) a primer having 70% to 100% identity with a sequence of SEQ ID NO:4,
(ii) a primer having 70% to 100% identity with a sequence of SEQ ID NO:5, and,
(iii) a probe having 70% to 100% identity with a sequence of SEQ ID NO:6; and
c. the HPV 45 primer and probe set comprises
(i) a primer having 70% to 100% identity with a sequence of SEQ ID NO:7,
(ii) a primer having 70% to 100% identity with a sequence of SEQ ID NO:8 and,
(iii) a probe having 70% to 100% identity with a sequence of SEQ ID NO:9.

20. The kit according to claim 17, further comprising a control primer and probe set.

21. The kit according to claim 20, wherein the control primer and probe set comprises
a. a primer having 70% to 100% identity with a sequence of SEQ ID NO:16,
b. a primer having 70% to 100% identity with a sequence of SEQ ID NO:17, and,
c. a probe having 70% to 100% identity with a sequence of SEQ ID NO:18.

22. A method of genotyping a high-risk HPV, the method comprising:
a. hybridizing at least one nucleic acid primer or probe according to claim 1 to at least a portion of a target sequence in an E6/E7 region of a first high-risk HPV genome; and
b. detecting hybridization of the nucleic acid to the E6/E7 region of the first high-risk HPV genome.

23. The method of claim 22 wherein the nucleic acid primer or probe comprises a sequence having about 70% to 100% identity to a sequence selected from the group consisting of SEQ ID NO:1 to SEQ ID NO:12 and SEQ ID NO:16 to SEQ ID NO:21, or a complement thereof.

24. The method of claim 22 wherein:
a. a nucleic acid primer is hybridized to the target sequence; and
b. hybridization is detected by a method comprising performing an amplification reaction.

25. The method of claim 24 wherein:
a. the high-risk HPV is selected from the group consisting of HPV 16, HPV 18, and HPV 45; and
b. said amplification reaction generates an amplicon corresponding to a portion of the E6/E7 region of the HPV genome.

26. The method of claim 25 wherein the nucleic acid primer has about 70% to 100% identity across its entire length to a nucleotide sequence selected from the group consisting of: SEQ ID NO: 1; SEQ ID NO: 2; SEQ ID NO: 4; SEQ ID NO: 5; SEQ ID NO: 7; and SEQ ID NO: 8 or a complement thereof.

27. The method of claim 22, wherein a nucleic acid probe is hybridized to the target sequence, which said nucleic acid probe is optionally detectably labeled.

28. The method of claim 22, wherein the said method is performed in a real time multiplex PCR format.

29. The method of claim 28, wherein said method is capable of distinguishing between HPV 16, HPV 18, and HPV 45 infections, said method comprising:
a. providing a sample suspected of comprising a high-risk HPV;
b. contacting the sample with:
(i) a primer set capable of amplifying a portion of a target sequence of an E6/E7 region of the HPV 16 genome, but not an E6/E7 region of an HPV 18 or HPV 45 genome;
(ii) a primer set capable of amplifying a portion of a target sequence of an E6/E7 region of the HPV 18 genome, but not an E6/E7 region of an HPV 16 or HPV 45 genome;
(iii) a primer set capable of amplifying a portion of a target sequence of an E6/E7 region of the HPV 45 genome, but not an E6/E7 region of an HPV 16 or HPV 18 genome;
(iv) a detectably labeled nucleic acid probe capable of hybridizing to a portion of the target sequence of the E6/E7 region of the HPV 16 genome, but not an E6/E7 region of an HPV 18 or HPV 45 genome;
(v) a detectably labeled nucleic acid probe capable of hybridizing to a portion of the target sequence of the E6/E7 region of the HPV 18 genome, but not an E6/E7 region of an HPV 16 or HPV 45 genome; and
(vi) a detectably labeled nucleic acid probe capable of hybridizing to a portion the target sequence of the E6/E7 region of the HPV 45 genome, but not an E6/E7 region of an HPV 16 or HPV 18 genome,
wherein each nucleic acid probe has a different detectable label;
c. performing a nucleic acid amplification; and
d. detecting the presence or absence of each detectable label.

30. The method of claim 22, wherein said method is performed on a platform that is capable of replicating a method performed by QIAGEN's Rotor-Gene PCR instrument.

31. The method of claim 22, wherein said method is unaffected by the presence or absence of PreservCyt® (Hologic, Bedford, Mass.) and SurePath™ (Becton Dickinson, Sparks Md.).

32. A composition comprising a nucleic acid primer or probe according to claim 1 hybridized to a target sequence in the E6/E7 region of a high-risk HPV genome.

33. The composition of claim 32 wherein the high-risk HPV genome is selected from the group consisting of HPV 16, HPV 18, and HPV 45.

34. The composition of claim 33 wherein the nucleic acid primer or probe comprises a sequence having 70% to 100% identity with a sequence selected from the group consisting of SEQ ID NO:1 to SEQ ID NO:12 and SEQ ID NO:16 to SEQ ID NO:21, or a complement thereof.

35. A primer according to claim 1 comprising a 3′ terminal sequence capable of hybridizing to a sequence selected from the group consisting of:
(a) nucleotides 695 to 719 of SEQ ID NO: 13 or a complement thereof; and
(b) nucleotides 811 to 834 of SEQ ID NO. 13 or a complement thereof,
wherein said primer is an HPV16 E6/E7-specific primer.

36. A kit comprising at least one primer according to claim 35.

37. The kit of claim 36 comprising:
(a) a first primer comprising a 3′ terminal sequence capable of hybridizing to a complement of nucleotides 695 to 719 of SEQ ID NO: 13; and
(b) a second primer comprising a 3′ terminal sequence capable of hybridizing to nucleotides 811 to 834 of SEQ ID NO. 13.

38. A kit of claim 37 further comprising a nucleic acid probe capable of hybridizing to nucleotides 751 to 775 of SEQ ID NO. 13 or a complement thereof.

39. A primer according to claim 1 comprising a 3′ terminal sequence capable of hybridizing to a sequence selected from the group consisting of:
a. nucleotides 724 to 747 of SEQ ID NO: 14 or a complement thereof; and
b. nucleotides 837 to 860 of SEQ ID NO. 14 or a complement thereof
wherein said primer is an HPV18 E6/E7-specific primer.

40. A kit comprising at least one primer according to claim 39.

41. The kit of claim 40 comprising:
(a) a first primer comprising 3′ terminal sequence capable of hybridizing to a complement nucleotides 724 to 747 of SEQ ID NO: 14; and
(b) a second primer comprising a 3′ terminal sequence capable of hybridizing to nucleotides 837 to 860 of SEQ ID NO. 14.

42. The kit of claim 41 further comprising a probe capable of hybridizing to nucleotides 748 to 773 of SEQ ID NO. 14.

43. A primer according to claim 1 comprising a 3′ terminal sequence capable of hybridizing to a sequence selected from the group consisting of:
(a) nucleotides 112 to 135 of SEQ ID NO: 15 or a complement thereof;
(b) nucleotides 208 to 231 of SEQ ID NO. 15 or a complement thereof;
(c) nucleotides 402 to 425 of SEQ ID NO: 15 or a complement thereof; and
(d) nucleotides 546 to 569 of SEQ ID NO. 15 or a complement thereof,
wherein the primer is an HPV45 E6/E7-specific primer.

44. A kit comprising at least one primer according to claim 43.

45. The kit according to claim 44 comprising:
(a) a first primer pair comprising:
(i) a first primer comprising a 3′ terminal sequence capable of hybridizing to a complement of nucleotides 112 to 135 of SEQ ID NO: 15; and
(ii) a second primer comprising a 3′ terminal sequence capable of hybridizing to nucleotides 208 to 231 of SEQ ID NO. 15; and/or
(b) a second primer pair comprising:
(i) a third primer comprising a 3′ terminal sequence capable of hybridizing to a complement of nucleotides 402 to 425 of SEQ ID NO: 15; and
(ii) a fourth primer comprising a 3′ terminal sequence capable of hybridizing to nucleotides 546 to 569 of SEQ ID NO. 15.

46. The kit of claim 45 further comprising at least one nucleic acid probe selected from the group consisting of:
(i) a probe capable of hybridizing to nucleotides 136 to 165 of SEQ ID NO. 15 or a complement thereof; and
(ii) a probe capable of hybridizing to nucleotides 517 to 542 of SEQ ID NO. 15 or a complement thereof.

47. A nucleic acid probe according to claim 1, said probe capable of hybridizing to nucleotides 751 to 775 of SEQ ID NO. 13 or a complement thereof.

48. A nucleic acid probe according to claim 1, said probe capable of hybridizing to nucleotides 748 to 773 of SEQ ID NO. 14 or a complement thereof.

49. A nucleic acid probe according to claim 1, said probe capable of hybridizing to:
(a) nucleotides 136 to 165 of SEQ ID NO. 15 or a complement thereof; or
(b) nucleotides 517 to 542 of SEQ ID NO. 15 or a complement thereof.


SEQUENCE LISTING (21 sequences)

SEQ ID NO: 1 - DNA, 24 bp, Artificial Sequence
  pcr primer
agaaccggacagagcccattacaa

SEQ ID NO: 2 - DNA, 24 bp, Artificial Sequence
  pcr primer
gcacacaattcctagtgtgcccat

SEQ ID NO: 3 - DNA, 25 bp, Artificial Sequence
  pcr probe
acgcttcggttgtgcgtacaaagca

SEQ ID NO: 4 - DNA, 24 bp, Artificial Sequence
  pcr primer
tcatcaacatttaccagcccgacg

SEQ ID NO: 5 - DNA, 24 bp, Artificial Sequence
  pcr primer
gaaacagctgctggaatgctcgaa

SEQ ID NO: 6 - DNA, 26 bp, Artificial Sequence
  pcr probe
agccgaaccacaacgtcacacaatgt

SEQ ID NO: 7 - DNA, 24 bp, Artificial Sequence
  pcr primer
ttgacgatccaaagcaacgaccct

SEQ ID NO: 8 - DNA, 24 bp, Artificial Sequence
  pcr primer
cctctgtgcgttccaatgttgctt

SEQ ID NO: 9 - DNA, 30 bp, Artificial Sequence
  pcr probe
acaagctaccagatttgtgcacagaattga

SEQ ID NO: 10 - DNA, 24 bp, Artificial Sequence
  pcr primer
ttgttaataaggtgcctgcggtgc

SEQ ID NO: 11 - DNA, 24 bp, Artificial Sequence
  pcr primer
tgtttccctacgtctgcgaagtct

SEQ ID NO: 12 - DNA, 26 bp, Artificial Sequence
  pcr probe
atacatgttgtgaccaggcacggcaa

SEQ ID NO: 13 - DNA, 7904 bp, Human papillomavirus type 16
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacataatgtatatgtacataatgtaattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttcatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaa

SEQ ID NO: 14 - DNA, 7857 bp, Human papillomavirus type 18
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 15 - DNA, 7858 bp, Human papillomavirus type 45
aatacttttaacaattatactacataaaaaagggtgtaaccgaaaacggttgcaaccaaaaacggtgcatataaaagctttgtggaaaagtgcattacaggatggcgcgctttgacgatccaaagcaacgaccctacaagctaccagatttgtgcacagaattgaatacatcactacaagacgtatctattgcctgtgtatattgcaaagcaacattggaacgcacagaggtatatcaatttgcttttaaagatttatgtatagtgtatagagactgtatagcatatgctgcatgccataaatgtatagacttttattccagaattagagaattaagatattattcaaactctgtatatggagagacactggaaaaaataactaatacagagttgtataatttgttaataaggtgcctgcggtgccagaaaccattgaacccagcagaaaaacgtagacaccttaaggacaaacgaagatttcacagcatagctggacagtaccgagggcagtgtaatacatgttgtgaccaggcacggcaagaaagacttcgcagacgtagggaaacacaagtatagcaataagtatgcatggaccccgggaaacactgcaagaaattgtattgcatttggaacctcagaatgaattagatcctgttgacctgttgtgttacgagcaattaagcgagtcagaggaggaaaacgatgaagcagatggagttagtcatgcacaactaccagcccgacgagccgaaccacagcgtcacaaaattttgtgtgtatgttgtaagtgtgacggcagaattgagcttacagtagagagctcggcagaggaccttagaacactacagcagctgtttttgagcaccttgtcctttgtgtgtccgtggtgtgcaactaaccaataatctacaatggcggatccagaaggtaccgacggggagggaacggggtgtaatggctggttctttgtagaaacaattgtagagaaaaaaacaggggatgtaatatcagatgatgaggatgaaactgcaacagatacagggtcggatatggtagattttattgacacacaattatccatttgtgaacaggcagagcaagagacagcacaggcattgttccatgcgcaggaagttcagaatgatgcacaggtgttgcatcttttaaaacgaaagtttgcaggaggcagcaaggaaaacagtccattaggggagcagctaagtgtggatacggatctaagtccacggttacaagaaatttcattaaatagtgggcacaaaaaagcaaaacgacggttgtttacaatatcagatagtggctatggctgttctgaagtggaagctgcagagactcaggtaactgtaaacactaatgcggaaaatggcggcagtgtacatagtacacaaagtagtggtggggatagtagtgacaatgcagaaaatgtagatccgcattgcagtattacagaactaaaggagctattacaagcaagtaacaaaaaggctgcaatgctggcagtatttaaagacatatatgggctgtcatttacggatttggttagaaattttaaaagtgataaaacaacatgtacagattgggtaatggctatatttggagttaatccaacggtagcagaaggctttaaaacattaattaaaccagcaacgttatacgcccatatccaatgtttagattgtaaatggggagtattaatattagctttattaagatataaatgtggcaaaaatagactaactgttgcaaaaggcttaagcacattgttgcacgtacctgaaacatgtatgttaattgaaccaccaaaattgcgaagtagtgttgcagcattatactggtatagaacaggtatatccaatattagtgaagtaagtggagacacacctgagtggatacaaagactgacaattattcaacatggtattgacgatagtaattttgatttgtcagacatggtgcaatgggcatttgataatgaccttacagatgaaagtgatatggcatttcaatatgcccaattagcagactgcaacagtaatgcagctgcatttttaaaaagtaactgccaagccaaatatttaaaagattgtgctgtaatgtgtagacattataaaagagcacaaaaacgccaaatgaatatgtctcaatggattaaatatagatgttccaaaatagatgaaggtggggattggagacccatagtacaattcctaagatatcagggagtagaatttattagctttttaagggcactaaaggaatttcttaaaggaacaccaaaaaaaaattgtatactgttatatggacctgcaaatacaggaaaatcgtattttggaatgagttttatacatttcctacaaggtgcaataatatcatttgtaaattcaaacagccatttttggttagaaccgttagcagatactaaggtagccatgttggatgatgccacacacacgtgttggacatattttgataattatatgagaaatgcattagatggtaatcctataagtatagacagaaagcataaaccattattacagctaaaatgtcctccaatcctattaacatccaatattgatccagcaaaagataataaatggccatatttagaaagtagggtgacggtatttacatttccacatgcatttccatttgataaaaatggtaatccagtatatgaaataaatgataaaaattggaaatgtttttttgaaaggacatggtccagattagatttgcacgaggacgatgaagatgcagacaccgaaggaatccctttcggaacgtttaagtgcgttacaggacaaaatactagaccactatgaaaatgacagtaaagacataaacagccaaataagttattggcaacttatacgtttggaaaatgcaatactatttacagcaagggaacatggtattaccaaactaaaccaccaggtggtgcctcctattaacatttcaaaaagcaaagcacataaagctattgaactgcaaatggccttaaagggccttgcacaaagcaagtataacaatgaggaatggacactgcaagatacatgcgaggaactatggaatacagaaccgtcgcagtgttttaaaaaaggcggtaaaaccgtgcacgtatactttgatggcaacaaggacaactgtatgaactatgtagtatgggacagtatatattatataactgagacagggatatgggacaaaacagcagcatgtgttagctattggggtgtatattatataaaagatggagataccacatattatgtacaatttaaaagcgaatgtgagaaatatggaaatagtaatacgtgggaagtacaatatgggggcaatgtaattgattgtaatgactctatgtgcagtaccagtgacgacacggtatccgctactcagattgttagacagctacaacacgcctccacgtcgacccccaaaaccgcatccgtgggcaccccaaaaccccacatccagacgccggctactaagcgacctagacagtgtggactcacagagcagcaccacggacgtgtcaacacccacgtgcacaacccgctcctgtgttcaagtacaagtaacaacaaaagaaggaaagtgtgtagtggtaacactacgcctataatacacttaaaaggtgacaaaaacagtttgaaatgtttaagatataggctacgcaaatatgcagaccattactcagaaatatcctccacctggcattggacaggttgtaataaaaacactggtatattaactgtaacatataatagtgaggtacaaagaaatacctttttggatgtagttactattcctaacagtgtacaaatctcggtgggatacatgactatatgaatctgtatattgtatacagtatgtaacattactatgctatctttagtgtttttattgtgcttttctgtgtgcctttatgtgtgctgcaatgtcccgcttgtgcagtctgtctatgtgtgtgcttttgcttggttgttggtgtttctttttatagttgttattacatccccattaacagcatttgctgtatacatttgttgctatttactacctatgtttgtattacatatgcatgctttacacaccatacaataattactataatgtacagtacagtgtaacatacctgtgatgtgcatgttgttgtatttttgtatttttgtatttttgtatttttgtattttatatgtttaataaaccatggtatcccaccgtgcagcacgtcgcaagcgggcctctgcaactgacttatatagaacatgtaagcaatccggtacgtgcccccctgatgttattaacaaagtggaaggcacaaccttagctgataaaattttacagtggtctagccttgggatatttttgggtggccttggcattggtaccggcagtggttctggaggccgtacgggctatgtacccttagggggcaggtctaatactgttgtggatgttggccccactaggccacctgtggttattgaacctgtagggcctactgatccatctattgttacgttggtagaggattccagtgttgttgcctctggtgctccggttcccacatttaccggaacctctgggtttgaaattacgtcttctggtactaccacaccagctgtgttggacatcacacctaccgtggactctgtttctatttcgtcaactagttttacaaatcctgcattttctgatccctctattattgaggtgccccaaacaggggaggtatcaggtaatatatttgttggtacaccaacatcgggcagccatggatatgaggaaatacctttacaaacatttgcatcttctgggtcaggtacggaacccattagtagtacccccctccctactgtgcggcgggtacggggtccccgcctgtatagtagggctaatcaacaggtccgtgtgtccacctcacagtttttaacacatccctcatcgttggttacatttgataatccagcttatgagcccctggacaccacactatcctttgagcctaccagtaatgttcctgattccgattttatggatattattcgtttgcataggccagcattatcctctagacgtggcactgttagatttagtagattgggtcaaagggcaaccatgtttacacgtagtggtaaacaaatagggggtagggtacatttttaccatgatataagccccattgctgctacagaggaaattgaattgcagcctttaattagtgctacaaatgatagtgacctgtttgatgtatatgcagacttcccacctcctgcgtccactacacctagcactatacacaaatcatttacatatccaaagtattccttgaccatgccttctactgctgcatcctcttacagtaatgttacagtaccattaacatctgcatgggatgtacctatatatactggcccggacattatattgccatcccatactcctatgtggcctagtacatctcctaccaatgcttccaccaccacctatataggtattcatggcacacaatattatttatggccatggtattattattttcctaaaaaacgtaaacgtattccctatttttttgcagatggctttgtggcggcctagtgacagtacggtatatcttccaccaccttctgtggccagagttgtcagcactgatgattatgtgtctcgcacaagcatattttatcatgcaggcagttcccgattattaactgtaggcaatccatattttagggttgtacctaatggtgcaggtaataaacaggctgttcctaaggtatccgcatatcagtatagggtgtttagagtagctttacccgatcctaataaatttggattacctgattctactatatataatcctgaaacacaacgtttggtttgggcatgtgtaggtatggaaattggtcgtgggcagcctttaggtattggcctaagtggccatccattttataataaattggatgatacagaaagtgctcatgcagctacagctgttattacgcaggatgttagggataatgtgtcagttgattataagcaaacacagctgtgtattttaggttgtgtacctgctattggtgagcactgggccaagggcacactttgtaaacctgcacaattgcaacctggtgactgtcctcctttggaacttaaaaacaccattattgaggatggtgatatggtggatacaggttatggggcaatggattttagtacattgcaggatacaaagtgcgaggttccattagacatttgtcaatccatctgtaaatatccagattatttgcaaatgtctgctgatccctatggggattctatgtttttttgcctacgccgtgaacaactgtttgcaagacatttttggaatagggcaggtgttatgggtgacacagtacctacggacctatatattaaaggcactagcgctaatatgcgtgaaacccctggcagttgtgtgtattccccttctcccagtggctctattattacttctgattctcaattatttaataagccatattggttacataaggcccagggccataacaatggtatttgttggcataatcagttgtttgttactgtagtggacactacccgcagtactaatttaacattatgtgcctctacacaaaatcctgtgccaagtacatatgaccctactaagtttaagcagtatagtagacatgtggaggaatatgatttacagtttatttttcagttgtgcactattactttaactgcagaggttatgtcatatatccatagtatgaatagtagtatattagaaaattggaattttggtgtccctccaccacctactacaagtttggtggatacatatcgttttgtgcaatcagttgctgttacctgtcaaaaggatactacacctccagaaaagcaggatccatatgataaattaaagttttggactgttgacctaaaggaaaaattttcctccgatttggatcaatatccccttggtcgaaagtttttagttcaggctgggttacgtcgtaggcctaccataggacctcgtaagcgtcctgctgcttccacgtctactgcatctactgcatctaggcctgccaaacgtgtacgtatacgtagtaagaaataatatgttagcacatatatgtatgtttgtatgtatggttttgtatgttgtatgtatgtatgtatttgtgtgatatattactgtattttgtttgtttgcgtgcgtgtatgtatgaatgtgccttgtggcatgtatggtgttactgtacataattgtggtattaaataaagtatgctaatagtgttgtgtagggttgcacccttgtgagtaacaatactatttgtgtgtatgtgtattgctttgtaccctatattctttcctgtatttcaagttataaacttgcatactacacagcatccattttacttataatcctccattttgctgtgcaaccgatttcggttgcctgtggcttatatgtgaccttttaaacataatacctaaactggcacatttacaacccctacatagtttaacctactggcgcgccttcttggcgtacatgtggcacacctggtattagtcattttcctgtccaggtgtactaaaacaatggcttgcacaactgtatccacaccctatgtaataaaactgcttttaggcacatattttagtctgtttttacctgtgctaattgtataattggcgtgtagaaccactttcttatccaacaatctgtctacttgttacataaactataaactgactcacttatacatacatagtttatgcaaccgaaaaaggttgggccctataacacataccttttctt

SEQ ID NO: 16 - DNA, 20 bp, Artificial Sequence
  pcr primer
gaagagccaaggacaggtac

SEQ ID NO: 17 - DNA, 20 bp, Artificial Sequence
  pcr primer
caacttcatccacgttcacc

SEQ ID NO: 18 - DNA, 23 bp, Artificial Sequence
  pcr probe
ccctagggttggccaatctactc

SEQ ID NO: 19 - DNA, 20 bp, Artificial Sequence
  pcr primer
acacaactgtgttcactagc

SEQ ID NO: 20 - DNA, 20 bp, Artificial Sequence
  pcr primer
caacttcatccacgttcacc

SEQ ID NO: 21 - DNA, 30 bp, Artificial Sequence
  pcr probe
tcaaacagacaccatggtgcatctgactcc